(19) United States
(12) Patent Application Publication
(10) Pub. No.: US 2003/0144292 A1
(43) Pub. Date: Jul. 31, 2003

(54) Carbocyclic side chain containing, N-substituted metalloprotease inhibitors

(76) Inventors: Michael George Natchus, Alpharetta, GA (US); Stanislaw Pikul, Germantown, MD (US); Neil Gregory Almstead, Holmdel, NJ (US); Matthew John Laufersweiler, Morrow, OH (US); Biswanath De, Cincinnati, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/246,224

(22) Filed: Sep. 18, 2002

Related U.S. Application Data
(63) Continuation-in-part of application No. PCT/US01/08782, filed on Mar. 20, 2001.
(60) Provisional application No. 60/190,993, filed on Mar. 21, 2000.

(51) Int. Cl. A61K 31/537; A61K 31/4172; A61K 31/403; A61K 31/4015; A61K 31/195; C07D 265/30

(52) U.S. Cl. 514/237.5; 514/409; 514/561; 514/424; 514/389; 544/159; 548/315.1; 548/408; 548/543; 562/430

(57) ABSTRACT

The compounds have a structure according to the following Formula (I):

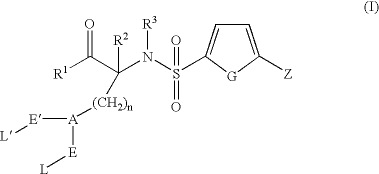

are effective in treating conditions characterized by excess activity of metalloprotease enzymes.

### CROSS REFERENCE

This application is a Continuation In Part of International Application PCT/US01/08782, with an international filing date of Mar. 20, 2001, which claims benefit of Provisional Application Serial No. 60/190,993, filed Mar. 21, 2000.

### TECHNICAL FIELD

This invention is directed to compounds which are useful in treating diseases associated with metalloprotease activity, particularly zinc metalloprotease activity. The invention is also directed to pharmaceutical compositions comprising the compounds, and to methods of treating metalloprotease-related maladies using the compounds or the pharmaceutical compositions.

### BACKGROUND

A number of structurally related metalloproteases effect the breakdown of structural proteins. These metalloproteases often act on the intercellular matrix, and thus are involved in tissue breakdown and remodeling. Such proteins are referred to as metalloproteases or MPs.

There are several different families of MPs, classified by sequence homology, disclosed in the art. These MPs include Matrix-Metallo Proteases (MMPs); zinc metalloproteases; many of the membrane bound metalloproteases; TNF converting enzymes; angiotensin-converting enzymes (ACEs); disintegrins, including ADAMs (see Wolfsberg et al, 131 J. Cell Bio. 275-78 October, 1995); and the enkephalinases. Examples of MPs include human skin fibroblast collagenase, human skin fibroblast gelatinase, human sputum collagenase, aggrecanse and gelatinase, and human stromelysin. Collagenases, stromelysin, aggrecanase and related enzymes are thought to be important in mediating the symptomatology of a number of diseases.

Potential therapeutic indications of MP inhibitors have been discussed in the literature. See, for example, U.S. Pat. No. 5,506,242 (Ciba Geigy Corp.) and U.S. Pat. No. 5,403,952 (Merck & Co.); the following PCT published applications: WO 96/06074 (British Bio Tech Ltd.); WO 96/00214 (Ciba Geigy), WO 95/35275 (British Bio Tech Ltd.), WO 95/35276 (British Bio Tech Ltd.), WO 95/33731 (Hoffman-LaRoche), WO 95/33709 (Hoffman-LaRoche), WO 95/32944 (British Bio Tech Ltd.), WO 95/26989 (Merck), WO 9529892 (DuPont Merck), WO 95/24921 (Inst. Opthamology), WO 95/23790 (SmithKline Beecham), WO 95/22966 (Sanofi Winthrop), WO 95/19965 (Glycomed), WO 95 19956 (British Bio Tech Ltd), WO 95/19957 (British Bio Tech Ltd.), WO 95/19961 (British Bio Tech Ltd.), WO 95/13289 (Chiroscience Ltd.), WO 95/12603 (Syntex), WO 95/09633 (Florida State Univ.), WO 95/09620 (Florida State Univ.), WO 95/04033 (Celltech), WO 94/25434 (Celltech), WO 94/25435 (Celltech); WO 93/14112 (Merck), WO 94/0019 (Glaxo), WO 93/21942 (British Bio Tech Ltd.), WO 92/22523 (Res. Corp. Tech Inc.), WO 94/10990 (British Bio Tech Ltd.), WO 93/09090 (Yamanouchi); British patents GB 2282598 (Merck) and GB 2268934 (British Bio Tech Ltd.); published European Patent Applications EP 95/684240 (Hoffman LaRoche), EP 574758 (Hoffman LaRoche) and EP 575844 (Hoffman LaRoche); published Japanese applications JP 08053403 (Fujusowa Pharm. Co. Ltd.) and JP 7304770 (Kanebo Ltd.); and Bird et al., J. Med. Chem., vol. 37, pp. 158-69 (1994).

Examples of potential therapeutic uses of MP inhibitors include rheumatoid arthritis—Mullins, D. E., et al., Biochim. Biophys. Acta. (1983) 695:117-214; osteoarthritis—Henderson, B., et al., Drugs of the Future (1990) 15:495-508; cancer—Yu, A. E. et al., Matrix Metalloproteinases—Novel Targets for Directed Cancer Therapy, Drugs & Aging, Vol. 11(3), p. 229-244 (September 1997), Chambers, A. F. and Matrisian, L. M., Review: Changing Views of the Role of Matrix Metalloproteinases in Metastasis, J. of the Nat'l Cancer Inst., Vol. 89(17), p. 1260-1270 (September 1997), Bramhall, S. R., The Matrix Metalloproteinases and Their Inhibitors in Pancreatic Cancer, Internat'l J. of Pancreatology, Vol. 4, p. 1101-1109 (May 1998), Nemunaitis, J. et al., Combined Analysis of Studies of the Effects of the Matrix Metalloproteinase Inhibitor Marimastat on Serum Tumor Markers in Advanced Cancer: Selection of a Biologically Active and Tolerable Dose for Longer-term Studies, Clin. Cancer Res., Vol 4, p. 1101-1109 (May 1998), and Rasmussen, H. S. and McCann, P. P, Matrix Metalloproteinase Inhibition as a Novel Anticancer Strategy: A Review with Special Focus on Batimastat and Marimastat, Pharmacol. Ther., Vol 75(1), p. 69-75 (1997); the metastasis of tumor cells—ibid, Broadhurst, M. J., et al., European Patent Application 276,436 (published 1987), Reich, R., et al., Cancer Res., Vol. 48, p. 3307-3312 (1988); multiple sclerosis—Gijbels et al., J. Clin. Invest., vol. 94, p. 2177-2182 (1994); and various ulcerations or ulcerative conditions of tissue. For example, ulcerative conditions can result in the cornea as the result of alkali burns or as a result of infection by Pseudomonas aeruginosa, Acanthamoeba, Herpes simplex and vaccinia viruses. Other examples of conditions characterized by undesired metalloprotease activity include periodontal disease, epidermolysis bullosa, fever, inflammation and scleritis (e.g., DeCicco et al., PCT published application WO 95/29892, published Nov. 9, 1995).

In view of the involvement of such metalloproteases in a number of disease conditions, attempts have been made to prepare inhibitors to these enzymes. A number of such inhibitors are disclosed in the literature. Examples include U.S. Pat. No. 5,183,900, issued Feb. 2, 1993 to Galardy; U.S. Pat. No. 4,996,358, issued Feb. 26, 1991 to Handa et al.; U.S. Pat. No. 4,771,038, issued Sep. 13, 1988 to Wolanin et al.; U.S. Pat. No. 4,743,587, issued May 10, 1988 to Dickens et al., European Patent Publication No. 575,844, published Dec. 29, 1993 by Broadhurst et al.; International Patent Publication No. WO 93/09090, published May 13, 1993 by Isomura et al.; World Patent Publication 92/17460, published Oct. 15, 1992 by Markwell et al.; and European Patent Publication No. 498,665, published Aug. 12, 1992 by Beckett et al.

It would be advantageous to inhibit these metalloproteases in treating diseases related to unwanted metalloprotease activity. Though a variety of MP inhibitors have been prepared, there is a continuing need for potent matrix metalloprotease inhibitors useful in treating diseases associated with metalloprotease activity.

### SUMMARY OF THE INVENTION

The invention provides compounds which are potent inhibitors of metalloproteases and which are effective in treating conditions characterized by excess activity of these enzymes. In particular, the present invention relates to compounds having a structure according to the following Formula (I):

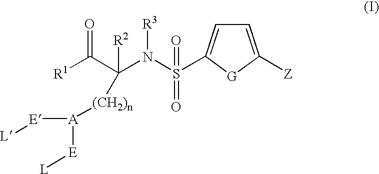

wherein:

(A) R¹ is selected from —OH and —NHOH;

(B) R² is selected from hydrogen, alkyl, alkenyl, alkynyl, heteroalkyl, haloalkyl, cycloalkyl, heterocycloalkyl, aryl, arylalkyl, heteroaryl and heteroarylalkyl; or R² and A form a ring as described in (D);

(C) R³ is selected from alkyl, alkenyl, alkynyl, heteroalkyl, haloalkyl, cycloalkyl, heterocycloalkyl, hydroxyl, alkoxy, heteroalkoxy, aryloxy, heteroaryloxy, aryl, arylalkyl, heteroaryl and heteroarylalkyl;

(D) A is a substituted or unsubstituted, monocyclic cycloalkyl having from 3 to 8 ring atoms; or A is bonded to R² where, together, they form a substituted or unsubstituted, monocyclic cycloalkyl having from 3 to 8 ring atoms;

(E) E and E′ are bonded to the same or different ring carbon atoms of A and are independently selected from a covalent bond, C₁-C₄ alkyl, aryl, heteroaryl, heteroalkyl, —O—, —S—, —N(R⁴)—, ═N, C═O, —C(═O)O—, —C(═O)N(R⁴)—, —SO₂— and —C(═S)N(R⁴)—, where R⁴ is selected from hydrogen, alkyl, alkenyl, alkynyl, heteroalkyl, haloalkyl, cycloalkyl, heterocycloalkyl, aryl, arylalkyl, heteroaryl and heteroarylalkyl; or R⁴ and L join to form a ring as described in (F)(2);

(F) (1) L and L′ are independently selected from hydrogen, alkyl, alkenyl, alkynyl, heteroalkyl, haloalkyl, aryl, arylalkyl, heteroaryl, heteroarylalkyl, cycloalkyl, heterocycloalkyl, —C(═O)R⁵, —C(═O)OR⁵, —C(═O)NR⁵R^{5′} and —SO₂R⁵, where R⁵ and R^{5′} each is independently selected from hydrogen, alkyl, alkenyl, alkynyl, heteroalkyl, haloalkyl, cycloalkyl, heterocycloalkyl, aryl, arylalkyl, heteroaryl and heteroarylalkyl; or

(2) L and R⁴ join to form an optionally substituted heterocyclic ring containing from 3 to 8 ring atoms of which from 1 to 3 are heteroatoms; or

(3) L and L′ join to form an optionally substituted cycloalkyl containing from 3 to 8 ring atoms or an optionally substituted hetercycloalkyl containing from 3 to 8 ring atoms of which from 1 to 3 are heteroatoms;

(G) G is selected from —S—, —O—, —N(R⁶)—, —C(R⁶)═C(R^{6′})—, —N═C(R⁶)— and —N═N—, where R⁶ and R^{6′} each is independently selected from hydrogen, alkyl, alkenyl, alkynyl, heteroalkyl, aryl, heteroaryl, cycloalkyl and heterocycloalkyl; and

(H) Z is selected from:

(1) cycloalkyl and heterocycloalkyl;

(2) -J-(CR⁷R^{7′})ₐR⁸ where:

(a) a is from 0 to about 4;

(b) J is selected from —C≡C—, —CH═CH—, —N═N—, —O—, —S— and —SO₂—;

(c) each R⁷ and R^{7′} is independently selected from hydrogen, alkyl, alkenyl, alkynyl, aryl, heteroalkyl, heteroaryl, cycloalkyl, heterocycloalkyl, halogen, haloalkyl, hydroxy and alkoxy; and

(d) R⁸ is selected from hydrogen, aryl, heteroaryl, alkyl, alkenyl, alkynyl, heteroalkyl, haloalkyl, heterocycloalkyl and cycloalkyl; and, if J is —C≡C— or —CH═CH—, then R⁸ may also be selected from —CONR⁹R^{9′} where (i) R⁹ and R^{9′} are independently selected from hydrogen, alkyl, alkenyl, alkynyl, haloalkyl, heteroalkyl, aryl, heteroaryl, cycloalkyl and heterocycloalkyl, or (ii) R⁹ and R^{9′}, together with the nitrogen atom to which they are bonded, join to form an optionally substituted heterocyclic ring containing from 5 to 8 ring atoms of which from 1 to 3 are heteroatoms;

(3) —NR¹⁰R^{10′} where:

(a) R¹⁰ and R^{10′} each is independently selected from hydrogen, alkyl, alkenyl, alkynyl, heteroalkyl, haloalkyl, aryl, heteroaryl, cycloalkyl, heteroalkyl and —C(═O)-Q-(CR¹¹R^{11′})_{b}R¹² where:

(i) b is from 0 to about 4;

(ii) Q is selected from a covalent bond and —N(R¹³)—; and

(iii) each R¹¹ and R^{11′} is independently selected from hydrogen, alkyl, alkenyl, alkynyl, aryl, heteroalkyl, heteroaryl, cycloalkyl, heterocycloalkyl, halogen, haloalkyl, hydroxy and alkoxy; either (A) R¹² and R¹³ each is independently selected from hydrogen, alkyl, alkenyl, alkynyl, heteroalkyl, haloalkyl, aryl, heteroaryl, cycloalkyl and heterocycloalkyl, or (B) R¹² and R¹³, together with the atoms to which they are bonded, join to form an optionally substituted heterocyclic ring containing from 5 to 8 ring atoms of which from 1 to 3 are heteroatoms; or R¹⁰ and R¹³, together with the nitrogen atoms to which they are bonded, join to form an optionally substituted heterocyclic ring containing from 5 to 8 ring atoms of which from 2 to 3 are heteroatoms; or

(b) R¹⁰ and R^{10′}, together with the nitrogen atom to which they are bonded, join to form an optionally substituted heterocyclic ring containing from 5 to 8 ring atoms of which from 1 to 3 are heteroatoms; and

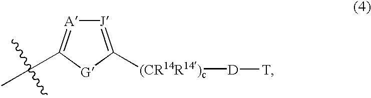

where:

(a) A′ and J′ are independently selected from —CH— and —N—;

(b) G′ is selected from —S—, —O—, —N(R¹⁵)—, —C(R¹⁵)═C(R^{15′})—, —N═C(R¹⁵)— and —N═N—, where R¹⁵ and R¹⁵ each is independently selected from hydrogen, alkyl, alkenyl, alkynyl, heteroalkyl, aryl, heteroaryl, cycloalkyl and heterocycloalkyl;

(c) c is from 0 to about 4;

(d) each R¹⁴ and R^{14′} is independently selected from hydrogen, alkyl, alkenyl, alkynyl, aryl, heteroalkyl, heteroaryl, cycloalkyl, heterocycloalkyl, halogen, haloalkyl, hydroxy and alkoxy;

(e) D is selected from a covalent bond, —O—, —SO_{d}—, —C(═O)—, C(═O)N(R¹⁶)—, —N(R¹⁶)—, and —N(R¹⁶)C(═O)—; where d is from 0 to 2 and R¹⁶ is selected from hydrogen, alkyl, alkenyl, alkynyl, aryl, heteroaryl, heteroalkyl, heteroaryl, cycloalkyl, heterocycloalkyl and haloalkyl; and

(f) T is —(CR¹⁷R^{17′})ₑ—R¹⁸ where e is from 0 to about 4; each R¹⁷ and R^{17′} is independently selected from hydrogen, alkyl, alkenyl, alkynyl, aryl, heteroalkyl, heteroaryl, cycloalkyl, heterocycloalkyl, halogen, haloalkyl, hydroxy, alkoxy and aryloxy; and R¹⁸ is selected from hydrogen, alkyl, alkenyl, alkynyl, halogen, heteroalkyl, haloalkyl, aryl, heteroaryl, cycloalkyl and heterocycloalkyl; or R¹⁷ and R¹⁸, together with the atoms to which they are bonded, join to form an optionally substituted heterocyclic ring containing from 5 to 8 atoms of which 1 to 3 are heteroatoms; or R¹⁶ and R¹⁸, together with the atoms to which they are bonded, join to form an optionally substituted heterocyclic ring containing from 5 to 8 atoms of which 1 to 3 are heteroatoms;

or an optical isomer, diastereomer or enantiomer for Formula (I), or a pharmaceutically-acceptable salt, or biohydrolyzable amide, ester, or imide thereof.

This invention also includes optical isomers, diastereomers and enantiomers of the formula above, and pharmaceutically-acceptable salts, biohydrolyzable amides, esters, and imides thereof.

The compounds of the present invention are useful for the treatment of diseases and conditions which are characterized by unwanted metalloprotease activity. Accordingly, the invention further provides pharmaceutical compositions comprising these compounds. The invention still further provides methods of treatment for metalloprotease-related maladies.

### DETAILED DESCRIPTION

I. Terms and Definitions:

The following is a list of definitions for terms used herein:

The following is a list of definitions for terms used herein.

“Acyl” or “carbonyl” is a radical formed by removal of the hydroxy from a carboxylic acid (i.e., R—C(═O)—). Preferred acyl groups include (for example) acetyl, formyl, and propionyl.

“Alkyl” is a saturated hydrocarbon chain having 1 to 15 carbon atoms, preferably 1 to 10, more preferably 1 to 4 carbon atoms. “Alkene” is a hydrocarbon chain having at least one (preferably only one) carbon-carbon double bond and having 2 to 15 carbon atoms, preferably 2 to 10, more preferably 2 to 4 carbon atoms. “Alkyne” is a hydrocarbon chain having at least one (preferably only one) carbon-carbon triple bond and having 2 to 15 carbon atoms, preferably 2 to 10, more preferably 2 to 4 carbon atoms. Alkyl, alkene and alkyne chains (referred to collectively as “hydrocarbon chains”) may be straight or branched and may be unsubstituted or substituted. Preferred branched alkyl, alkene and alkyne chains have one or two branches, preferably one branch. Preferred chains are alkyl. Alkyl, alkene and alkyne hydrocarbon chains each may be unsubstituted or substituted with from 1 to 4 substituents; when substituted, preferred chains are mono-, di-, or tri-substituted. Alkyl, alkene and alkyne hydrocarbon chains each may be substituted with halo, hydroxy, aryloxy (e.g., phenoxy), heteroaryloxy, acyloxy (e.g., acetoxy), carboxy, aryl (e.g., phenyl), heteroaryl, cycloalkyl, heterocycloalkyl, spirocycle, amino, amido, acylamino, keto, thioketo, cyano, or any combination thereof. Preferred hydrocarbon groups include methyl, ethyl, propyl, isopropyl, butyl, vinyl, allyl, butenyl, and exomethylenyl.

Also, as referred to herein, a “lower” alkyl, alkene or alkyne moiety (e.g., “lower alkyl”) is a chain comprised of 1 to 6, preferably from 1 to 4, carbon atoms in the case of alkyl and 2 to 6, preferably 2 to 4, carbon atoms in the case of alkene and alkyne.

“Alkoxy” is an oxygen radical having a hydrocarbon chain substituent, where the hydrocarbon chain is an alkyl or alkenyl (i.e., —O-alkyl or —O-alkenyl). Preferred alkoxy groups include (for example) methoxy, ethoxy, propoxy and allyloxy.

“Aryl” is an aromatic hydrocarbon ring. Aryl rings are monocyclic or fused bicyclic ring systems. Monocyclic aryl rings contain 6 carbon atoms in the ring. Monocyclic aryl rings are also referred to as phenyl rings. Bicyclic aryl rings contain from 8 to 17 carbon atoms, preferably 9 to 12 carbon atoms, in the ring. Bicyclic aryl rings include ring systems wherein one ring is aryl and the other ring is aryl, cycloalkyl, or heterocycloakyl. Preferred bicyclic aryl rings comprise 5-, 6- or 7-membered rings fused to 5-, 6-, or 7-membered rings. Aryl rings may be unsubstituted or substituted with from 1 to 4 substituents on the ring. Aryl may be substituted with halo, cyano, nitro, hydroxy, carboxy, amino, acylamino, alkyl, heteroalkyl, haloalkyl, phenyl, aryloxy, alkoxy, heteroalkyloxy, carbamyl, haloalkyl, methylenedioxy, heteroaryloxy, or any combination thereof. Preferred aryl rings include naphthyl, tolyl, xylyl, and phenyl. The most preferred aryl ring radical is phenyl.

“Aryloxy” is an oxygen radical having an aryl substituent (i.e., —O-aryl). Preferred aryloxy groups include (for example) phenoxy, napthyloxy, methoxyphenoxy, and methylenedioxyphenoxy.

“Cycloalkyl” is a saturated or unsaturated hydrocarbon ring. Cycloalkyl rings are not aromatic. Cycloalkyl rings are monocyclic, or are fused, spiro, or bridged bicyclic ring systems. Monocyclic cycloalkyl rings contain from about 3 to about 9 carbon atoms, preferably from 3 to 7 carbon atoms, in the ring. Bicyclic cycloalkyl rings contain from 7 to 17 carbon atoms, preferably from 7 to 12 carbon atoms, in the ring. Preferred bicyclic cycloalkyl rings comprise 4-, 5-, 6- or 7-membered rings fused to 5-, 6-, or 7-membered rings. Cycloalkyl rings may be unsubstituted or substituted with from 1 to 4 substituents on the ring. Cycloalkyl may be substituted with halo, cyano, alkyl, heteroalkyl, haloalkyl, phenyl, keto, hydroxy, carboxy, amino, acylamino, aryloxy, heteroaryloxy, or any combination thereof. Preferred cycloalkyl rings include cyclopropyl, cyclopentyl, and cyclohexyl.

“Halo” or “halogen” is fluoro, chloro, bromo or iodo. Preferred halo are fluoro, chloro and bromo; more preferred typically are chloro and fluoro, especially fluoro.

“Haloalkyl” is a straight, branched, or cyclic hydrocarbon substituted with one or more halo substituents. Preferred are C₁-C₁₂ haloalkyls; more preferred are C₁-C₆ haloalkyls; still more preferred still are C₁-C₃ haloalkyls. Preferred halo substituents are fluoro and chloro. The most preferred haloalkyl is trifluoromethyl.

“Heteroatom” is a nitrogen, sulfur, or oxygen atom. Groups containing more than one heteroatom may contain different heteroatoms.

“Heteroalkyl” is a saturated or unsaturated chain containing carbon and at least one heteroatom, wherein no two heteroatoms are adjacent. Heteroalkyl chains contain from 2 to 15 member atoms (carbon and heteroatoms) in the chain, preferably 2 to 10, more preferably 2 to 5. For example, alkoxy (i.e., —O-alkyl or —O-heteroalkyl) radicals are included in heteroalkyl. Heteroalkyl chains may be straight or branched. Preferred branched heteroalkyl have one or two branches, preferably one branch. Preferred heteroalkyl are saturated. Unsaturated heteroalkyl have one or more carbon-carbon double bonds and/or one or more carbon-carbon triple bonds. Preferred unsaturated heteroalkyls have one or two double bonds or one triple bond, more preferably one double bond. Heteroalkyl chains may be unsubstituted or substituted with from 1 to 4 substituents. Preferred substituted heteroalkyl are mono-, di-, or tri-substituted. Heteroalkyl may be substituted with lower alkyl, haloalkyl, halo, hydroxy, aryloxy, heteroaryloxy, acyloxy, carboxy, monocyclic aryl, heteroaryl, cycloalkyl, heterocycloalkyl, spirocycle, amino, acylamino, amido, keto, thioketo, cyano, or any combination thereof.

“Heteroaryl” is an aromatic ring containing carbon atoms and from 1 to about 6 heteroatoms in the ring. Heteroaryl rings are monocyclic or fused bicyclic ring systems. Monocyclic heteroaryl rings contain from about 5 to about 9 member atoms (carbon and heteroatoms), preferably 5 or 6 member atoms, in the ring. Bicyclic heteroaryl rings contain from 8 to 17 member atoms, preferably 8 to 12 member atoms, in the ring. Bicyclic heteroaryl rings include ring systems wherein one ring is heteroaryl and the other ring is aryl, heteroaryl, cycloalkyl, or heterocycloalkyl. Preferred bicyclic heteroaryl ring systems comprise 5-, 6- or 7-membered rings fused to 5-, 6-, or 7-membered rings. Heteroaryl rings may be unsubstituted or substituted with from 1 to 4 substituents on the ring. Heteroaryl may be substituted with halo, cyano, nitro, hydroxy, carboxy, amino, acylamino, alkyl, heteroalkyl, haloalkyl, phenyl, alkoxy, aryloxy, heteroaryloxy, or any combination thereof. Preferred heteroaryl rings include, but are not limited to, the following:

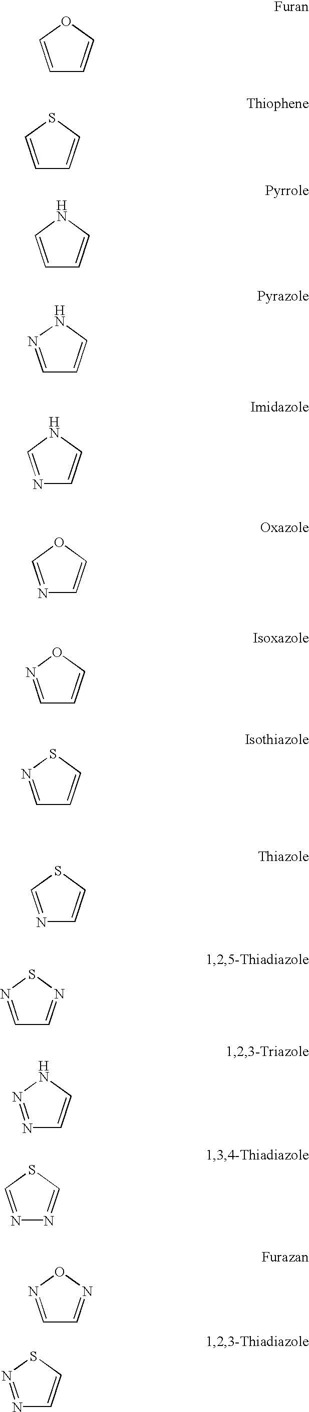

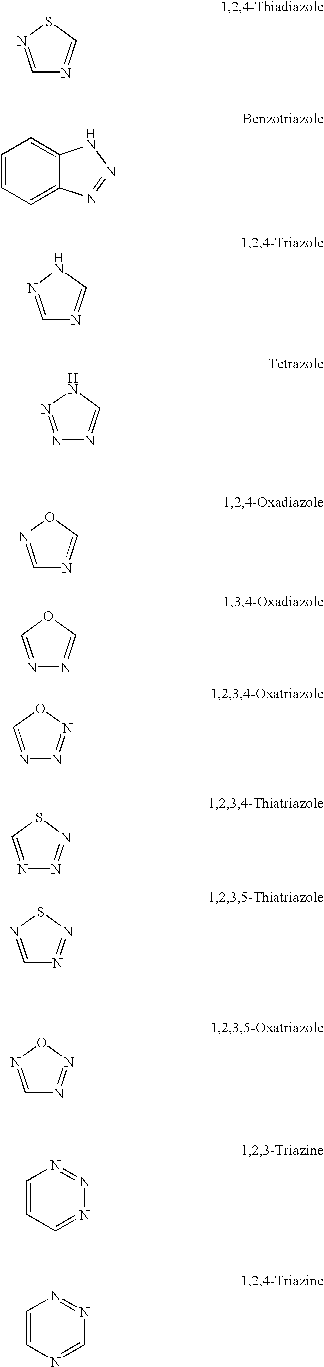

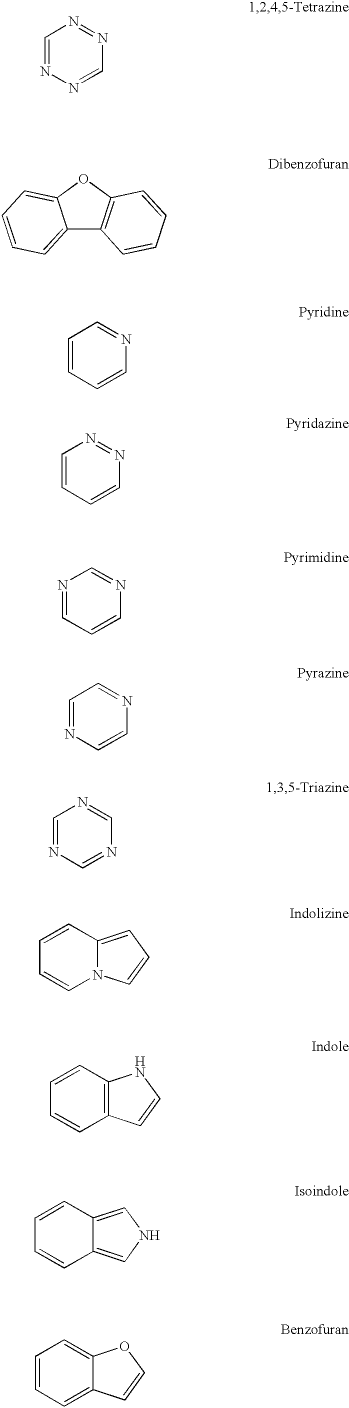

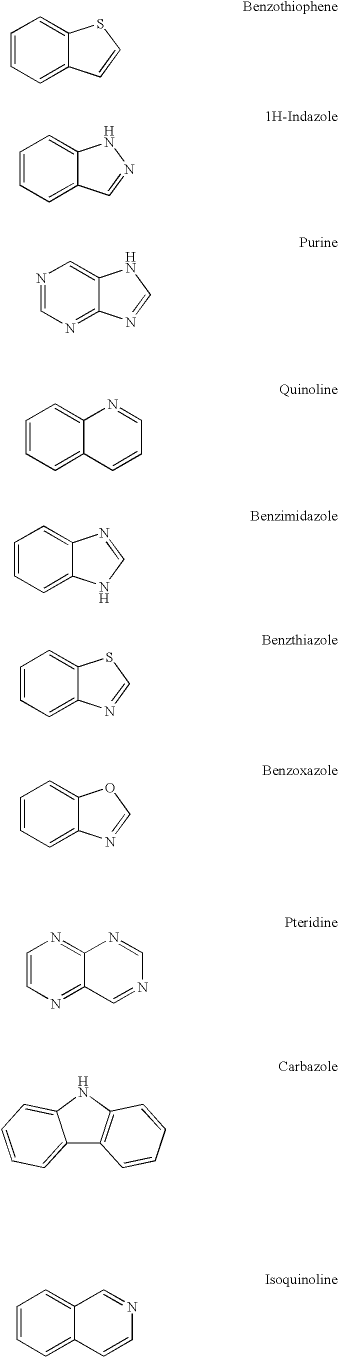

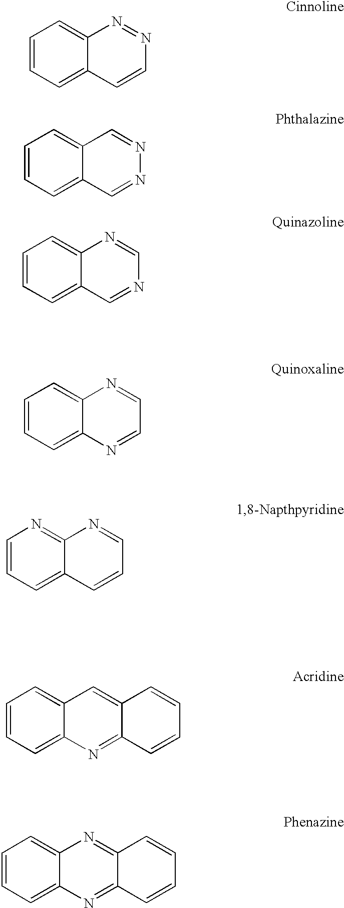

“Heteroaryloxy” is an oxygen radical having a heteroaryl substituent (i.e., —O-heteroaryl). Preferred heteroaryloxy groups include (for example) pyridyloxy, furanyloxy, (thiophene)oxy, (oxazole)oxy, (thiazole)oxy, (isoxazole)oxy, pyrmidinyloxy, pyrazinyloxy, and benzothiazolyloxy.

“Heterocycloalkyl” is a saturated or unsaturated ring containing carbon atoms and from 1 to about 4 (preferably 1 to 3) heteroatoms in the ring. Heterocycloalkyl rings are not aromatic. Heterocycloalkyl rings are monocyclic, or are fused, bridged, or Spiro bicyclic ring systems. Monocyclic heterocycloalkyl rings contain from about 3 to about 9 member atoms (carbon and heteroatoms), preferably from 5 to 7 member atoms, in the ring. Bicyclic heterocycloalkyl rings contain from 7 to 17 member atoms, preferably 7 to 12 member atoms, in the ring. Bicyclic heterocycloalkyl rings contain from about 7 to about 17 ring atoms, preferably from 7 to 12 ring atoms. Bicyclic heterocycloalkyl rings may be fused, spiro, or bridged ring systems. Preferred bicyclic heterocycloalkyl rings comprise 5-, 6- or 7-membered rings fused to 5-, 6-, or 7-membered rings. Heterocycloalkyl rings may be unsubstituted or substituted with from 1 to 4 substituents on the ring. Heterocycloalkyl may be substituted with halo, cyano, hydroxy, carboxy, keto, thioketo, amino, acylamino, acyl, amido, alkyl, heteroalkyl, haloalkyl, phenyl, alkoxy, aryloxy or any combination thereof. Preferred substituents on heterocycloalkyl include halo and haloalkyl. Preferred heterocycloalkyl rings include, but are not limited to, the following:

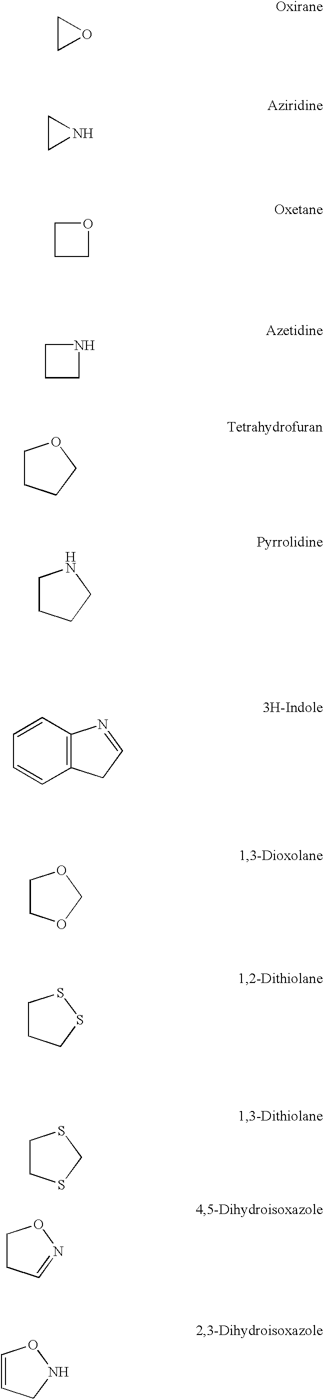

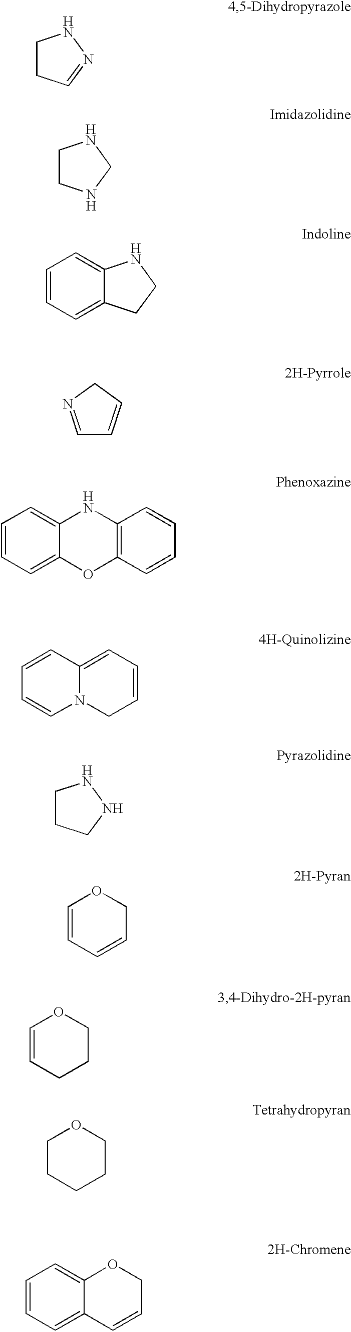

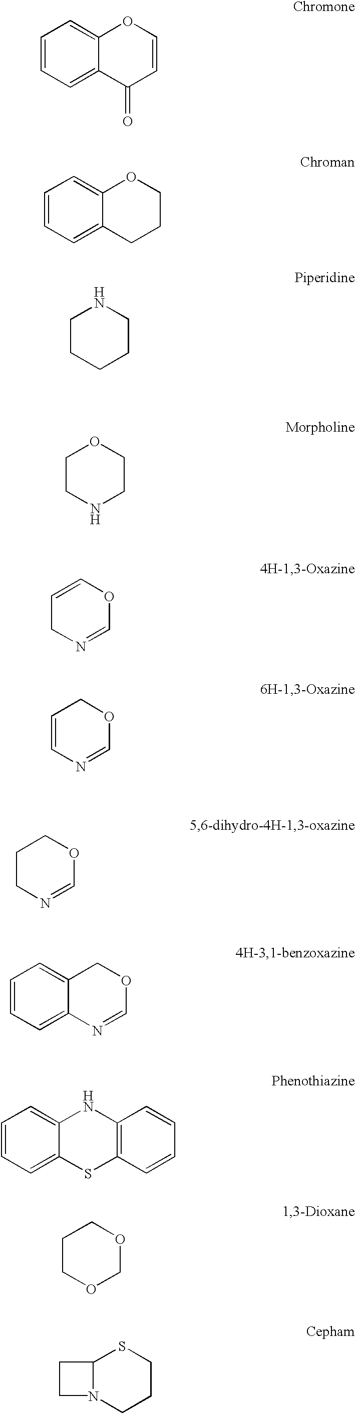

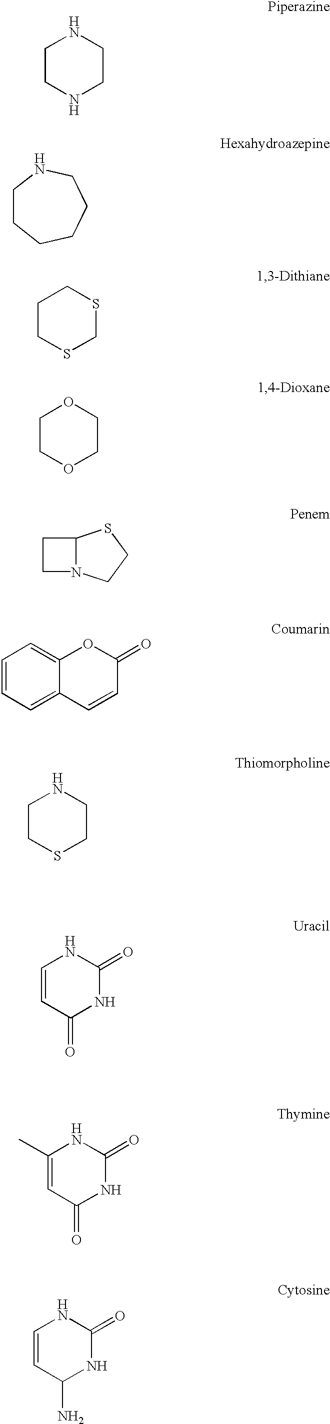

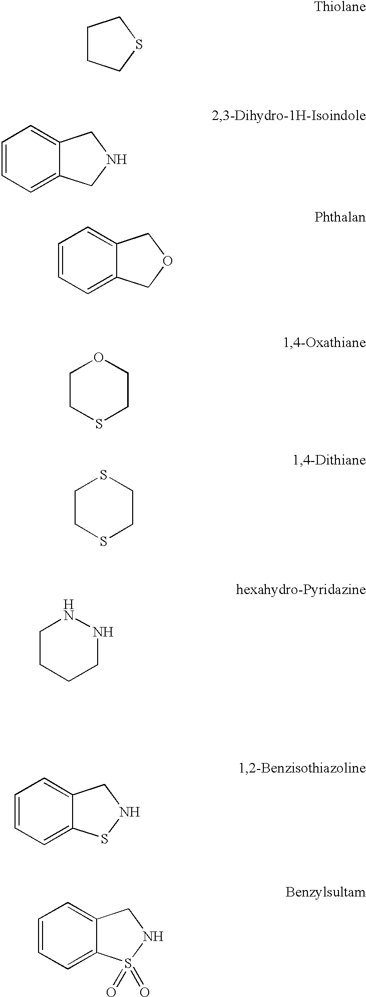

As used herein, “mammalian metalloprotease” refers to the proteases disclosed in the “Background” section of this application. The compounds of the present invention are preferably active against “mammalian metalloproteases”, including any metal-containing (preferably zinc-containing) enzyme found in animal, preferably mammalian, sources capable of catalyzing the breakdown of collagen, gelatin or proteoglycan under suitable assay conditions. Appropriate assay conditions can be found, for example, in U.S. Pat. No. 4,743,587, which references the procedure of Cawston, et al., Anal. Biochem. (1979) 99:340-345; use of a synthetic substrate is described by Weingarten, H., et al., Biochem. Biophy. Res. Comm. (1984) 139:1184-1187. See also Knight, C. G. et al., “A Novel Coumarin-Labelled Peptide for Sensitive Continuous Assays of the Matrix Metalloproteases”, FEBS Letters, Vol. 296, pp. 263-266 (1992). Any standard method for analyzing the breakdown of these structural proteins can, of course, be used. The present compounds are more preferably active against metalloprotease enzymes that are zinc-containing proteases which are similar in structure to, for example, human stromelysin or skin fibroblast collagenase. The ability of candidate compounds to inhibit metalloprotease activity can, of course, be tested in the assays described above. Isolated metalloprotease enzymes can be used to confirm the inhibiting activity of the invention compounds, or crude extracts which contain the range of enzymes capable of tissue breakdown can be used.

“Spirocycle” is an alkyl or heteroalkyl diradical substituent of alkyl or heteroalkyl wherein said diradical substituent is attached geminally and wherein said diradical substituent forms a ring, said ring containing 4 to 8 member atoms (carbon or heteroatom), preferably 5 or 6 member atoms.

While alkyl, heteroalkyl, cycloalkyl, and heterocycloalkyl groups may be substituted with hydroxy, amino, and amido groups as stated above, the following are not envisioned in the invention:

1. Enols (OH attached to a carbon bearing a double bond).

2. Amino groups attached to a carbon bearing a double bond (except for vinylogous amides).

3. More than one hydroxy, amino, or amido attached to a single carbon (except where two nitrogen atoms are attached to a single carbon atom and all three atoms are member atoms within a heterocycloalkyl ring).

4. Hydroxy, amino, or amido attached to a carbon that also has a heteroatom attached to it.

5. Hydroxy, amino, or amido attached to a carbon that also has a halogen attached to it.

A “pharmaceutically-acceptable salt” is a cationic salt formed at any acidic (e.g., hydroxamic or carboxylic acid) group, or an anionic salt formed at any basic (e.g., amino) group. Many such salts are known in the art, as described in World Patent Publication 87/05297, Johnston et al., published Sep. 11, 1987 incorporated by reference herein. Preferred cationic salts include the alkali metal salts (such as sodium and potassium), and alkaline earth metal salts (such as magnesium and calcium) and organic salts. Preferred anionic salts include the halides (such as chloride salts), sulfonates, carboxylates, phosphates, and the like.

Such salts are well understood by the skilled artisan, and the skilled artisan is able to prepare any number of salts given the knowledge in the art. Furthermore, it is recognized that the skilled artisan may prefer one salt over another for reasons of solubility, stability, formulation ease and the like. Determination and optimization of such salts is within the purview of the skilled artisan's practice.

A “biohydrolyzable amide” is an amide of a hydroxamic acid-containing (i.e., R¹ in Formula (I) is —NHOH) metalloprotease inhibitor that does not interfere with the inhibitory activity of the compound, or that is readily converted in vivo by an animal, preferably a mammal, more preferably a human subject, to yield an active metalloprotease inhibitor. Examples of such amide derivatives are alkoxyamides, where the hydroxyl hydrogen of the hydroxamic acid of Formula (I) is replaced by an alkyl moiety, and acyloxyamides, where the hydroxyl hydrogen is replaced by an acyl moiety (i.e., R—C(═O)—).

A “biohydrolyzable hydroxy imide” is an imide of a hydroxamic acid-containing metalloprotease inhibitor that does not interfere with the metalloprotease inhibitory activity of these compounds, or that is readily converted in vivo by an animal, preferably a mammal, more preferably a human subject to yield an active metalloprotease inhibitor. Examples of such imide derivatives are those where the amino hydrogen of the hydroxamic acid of Formula (I) is replaced by an acyl moiety (i.e., R—C(═O)—).

A “biohydrolyzable ester” is an ester of a carboxylic acid-containing (i.e., R¹ in Formula (I) is —OH) metalloprotease inhibitor that does not interfere with the metalloprotease inhibitory activity of these compounds or that is readily converted by an animal to yield an active metalloprotease inhibitor. Such esters include lower alkyl esters, lower acyloxy-alkyl esters (such as acetoxymethyl, acetoxyethyl, aminocarbonyloxymethyl, pivaloyloxymethyl and pivaloyloxyethyl esters), lactonyl esters (such as phthalidyl and thiophthalidyl esters), lower alkoxyacyloxyalkyl esters (such as methoxycarbonyloxymethyl, ethoxycarbonyloxyethyl and isopropoxycarbonyloxyethyl esters), alkoxyalkyl esters, choline esters and alkyl acylamino alkyl esters (such as acetamidomethyl esters).

A “solvate” is a complex formed by the combination of a solute (e.g., a metalloprotease inhibitor) and a solvent (e.g., water). See J. Honig et al., The Van Nostrand Chemist's Dictionary, p. 650 (1953). Pharmaceutically-acceptable solvents used according to this invention include those that do not interfere with the biological activity of the metalloprotease inhibitor (e.g., water, ethanol, acetic acid, N,N-dimethylformamide and others known or readily determined by the skilled artisan).

The terms “optical isomer”, “stereoisomer”, and “diastereomer” have the standard art recognized meanings (see, e.g., Hawley's Condensed Chemical Dictionary, 11th Ed.). The illustration of specific protected forms and other derivatives of the compounds of the instant invention is not intended to be limiting. The application of other useful protecting groups, salt forms, etc. is within the ability of the skilled artisan.

II. Compounds:

The subject invention involves compounds of Formula (I):

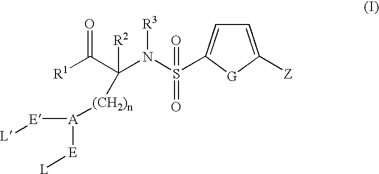

where R¹, R², R³, n, A, E, E′, L, L′, G and Z have the meanings described above. The following provides a description of particularly preferred moieties, but is not intended to limit the scope of the claims.

R¹ is selected from —OH and —NHOH, preferably —OH.

R² is selected from hydrogen, alkyl, alkenyl, alkynyl, heteroalkyl, haloalkyl, cycloalkylalkyl, heterocycloalkylalkyl, arylalkyl and heteroarylalkyl; preferably hydrogen or alkyl, more preferably hydrogen.

R³ is selected from alkyl, alkenyl, alkynyl, heteroalkyl, haloalkyl, cycloalkyl, heterocycloalkyl, hydroxyl, alkoxy, heteroalkoxy, aryloxy, heteroaryloxy, aryl, arylalkyl, heteroaryl and heteroarylalkyl. R³ is preferably alkyl, heteroalkyl, cycloalkyl, heterocycloalkyl, aryl, arylalkyl, heteroaryl and heteroarylalkyl.

n is from 0 to about 4, preferably 0 or 1, more preferably 0.

A is a substituted or unsubstituted, monocyclic cycloalkyl having from 3 to 8 ring atoms. Alternatively, A and R² can together form a substituted or unsubstituted, monocyclic cycloalkyl having from 3 to 8 ring atoms. A is preferably substituted or unsubstituted cyclopentane or cyclohexane.

E and E′ are bonded to the same or different ring carbon atoms of A and are independently selected from a covalent bond, C₁-C₄ alkyl, aryl, heteroaryl, heteroalkyl, —O—, —S—, —N(R⁴)—, ═N—, —C(═O)—, —C(═O)O—, —C(═O)N(R⁴)—, —SO₂— and —C(═S)N(R⁴)—. In those embodiments where L and R⁴ do not join to form a ring, E is preferably selected from —O—, —S—, NR⁴, or —SO₂—, more preferably E is —O— or NR⁴; and E′ is preferably a bond. In those embodiments where L and R⁴ join to form a ring, E is preferably NR⁴ and E′ is preferably a bond.

R⁴ and R^{4′} are independently selected from hydrogen, alkyl, alkenyl, alkynyl, heteroalkyl, haloalkyl, cycloalkyl, heterocycloalkyl, aryl, arylalkyl, heteroaryl and heteroarylalkyl. Preferred are hydrogen, alkyl, heteroalkyl, haloalkyl, cycloalkyl, heterocycloalkyl, aryl, arylalkyl, heteroaryl and heteroarylalkyl.

L and L′ are independently selected from hydrogen, alkyl, alkenyl, alkynyl, heteroalkyl, haloalkyl, aryl, arylalkyl, heteroaryl, heteroarylalkyl, cycloalkyl, heterocycloalkyl, —C(═O)R⁵, —C(═O)OR⁵, —C(═O)NR⁵R^{5′}, —SO₂R⁵. In those embodiments where L and R⁴ do not join to form a ring, L is preferably selected from hydrogen, alkyl, heteroalkyl, aryl, arylalkyl, heteroaryl, heteroarylalkyl, heterocycloalkyl, —C(═O)R⁵, —C(═O)OR⁵, —C(═O)NR⁵R^{5′}, —SO₂R⁵; and L′ is hydrogen. In those embodiments where L and R⁴ join to form a ring, L is preferably selected from alkyl, heteroalkyl, C(O)R⁵, C(O)OR⁵, C(O)NR⁵R^{5′}, SO₂R⁵; and L′ is hydrogen.

R⁵ and R^{5′} are independently selected from hydrogen, alkyl, alkenyl, alkynyl, heteroalkyl, haloalkyl, cycloalkyl, heterocycloalkyl, aryl, arylalkyl, heteroaryl and heteroarylalkyl. Preferred are hydrogen, alkyl, heteroalkyl, cycloalkyl, heterocycloalkyl, aryl, arylalkyl, heteroaryl and heteroarylalkyl.

Alternatively, L and R⁴ join to form an optionally substituted heterocyclic ring containing from 3 to 8 ring atoms of which from 1 to 3 are heteroatoms.

Alternatively, L and L′ join to form an optionally substituted cycloalkyl containing from 3 to 8 ring atoms or an optionally substituted hetercycloalkyl containing from 3 to 8 ring atoms of which from 1 to 3 are heteroatoms. In such embodiments, where E and E′ are bonded to the same ring carbon atom of A, the resulting ring is a spiro moiety on A. Preferred spiro moieties are heterocyclcoalkyls. In such embodiments, where E and E′ are bonded to different ring carbon atoms of A, the resulting ring is fused to A. Preferred fused rings are heterocycloalkyls.

G is selected from —S—, —O—, —N(R⁶)—, —C(R⁶)═C(R^{6′})—, —N═C(R⁶)—, and —N═N— and is preferably —S— or —C(R⁶)═C(R^{6′})—. R⁶ and R^{6′} each is independently selected from hydrogen, alkyl, alkenyl, alkynyl, heteroalkyl, aryl, heteroaryl, cycloalkyl, and heterocycloalkyl and preferably is hydrogen or alkyl.

Z is selected from cycloalkyl and heterocycloalkyl; -J-(CR⁷R^{7′})ₐR⁸; —NR¹⁰R^{10′}; and

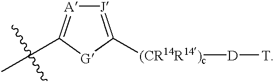

Preferred is where Z is -J-(CR⁷R^{7′})ₐR⁸; —NR¹⁰R^{10′}; and Most preferred is where Z is

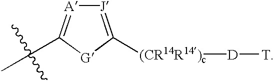

When Z is cycloalkyl or heterocycloalkyl, preferred is where Z is an optionally substituted piperidine or piperazine.

When Z is -J-(CR⁷R^{7′})ₐR⁸, a is from 0 to about 4, preferably 0 or 1. J is selected from —C≡C—, —CH═CH—, —N═N—, —O—, —S— and —SO₂—. Preferred is where J is —C≡C—, —CH═CH—, —N═N—, —O— or —S—; more preferred is —C≡C—, —CH═CH—, or —N═N—. Each R⁷ and R^{7′} each is independently selected from hydrogen, alkyl, alkenyl, alkynyl, aryl, heteroalkyl, heteroaryl, cycloalkyl, heterocycloalkyl, halogen, haloalkyl, hydroxy, and alkoxy preferably each R⁷ is hydrogen and each R^{7′} is independently hydrogen or lower alkyl. R⁸ is selected from aryl, heteroaryl, alkyl, alkenyl, alkynyl, heteroalkyl, haloalkyl, heterocycloalkyl and cycloalkyl; preferably R⁸ is aryl, heteroaryl, heterocycloalkyl or cycloalkyl. However, if J is —C≡C— or —CH═CH—, then R⁸ may also be selected from —C(═O)NR⁹R^{9′} where (i) R⁹ and R^{9′} are independently selected from hydrogen, alkyl, alkenyl, alkynyl, haloalkyl, heteroalkyl, aryl, heteroaryl, cycloalkyl, and heterocycloalkyl, or (ii) R⁹ and R^{9′}, together with the nitrogen atom to which they are bonded, join to form an optionally substituted heterocyclic ring containing from 5 to 8 (preferably 5 or 6) ring atoms of which from 1 to 3 (preferably 1 or 2) are heteroatoms.

When Z is —NR¹⁰R^{10′}, R¹⁰ and R^{10′} each is independently selected from hydrogen, alkyl, alkenyl, alkynyl, heteroalkyl, haloalkyl, aryl, heteroaryl, cycloalkyl, heteroalkyl, and —C(O)-Q-(CR¹¹R^{11′})_{b}R¹²; preferably R¹⁰ is hydrogen and R^{10′} is —C(O)-Q-(CR¹¹R^{11′})_{b}R¹². When R¹⁰ or R^{10′} is —C(O)-Q-(CR¹¹R^{11′})_{b}R¹², b is from 0 to about 4; b is preferably 0 or 1. Q is selected from a covalent bond and —N(R¹³)—; Q is preferably a covalent bond. Each R¹¹ and R^{11′} is independently selected from hydrogen, alkyl, alkenyl, alkynyl, aryl, heteroalkyl, heteroaryl, cycloalkyl, heterocycloalkyl, halogen, haloalkyl, hydroxy, and alkoxy; preferably each R¹¹ is hydrogen and each R^{11′} is independently hydrogen or lower alkyl. R¹² and R¹³ (i) each is independently selected from hydrogen, alkyl, alkenyl, alkynyl, heteroalkyl, haloalkyl, aryl, heteroaryl, cycloalkyl, and heterocycloalkyl, or (ii) R¹² and R¹³, together with the nitrogen atom to which they are bonded, join to form an optionally substituted heterocyclic ring containing from 5 to 8 (preferably 5 or 6) ring atoms of which from 1 to 3 (preferably 1 or 2) are heteroatoms; preferably R¹² is alkyl, aryl, heteroaryl, cycloalkyl or heterocycloalkyl. Alternatively, R¹⁰ and R¹³, together with the nitrogen atoms to which they are bonded, join to form an optionally substituted heterocyclic ring containing from 5 to 8 ring atoms of which from 1 to 3 are heteroatoms.

Alternatively, R¹⁰ and R^{10′}, together with the nitrogen atom to which they are bonded, join to form an optionally substituted heterocyclic ring containing from 5 to 8 (preferably 5 or 6) ring atoms of which from 1 to 3 (preferably 1 or 2) are heteroatoms.

When Z is

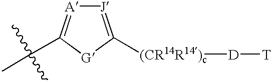

(referred to herein as Formula (A)), A′ and J′ are independently selected from —CH— and —N—; preferred is where A′ is —CH and J′ is —CH. G′ is selected from —S—, —O—, —N(R¹⁵)—, —C(R¹⁵)═C(R^{15′})—, —N═C(R¹⁵)—, and —N═N—; preferably —N═C(R¹⁵)— or —C(R¹⁵)═C(R^{15′})—. R¹⁵ and R^{15′} each is independently selected from hydrogen, alkyl, alkenyl, alkynyl, heteroalkyl, aryl, heteroaryl, cycloalkyl, and heterocycloalkyl; preferably hydrogen or lower alkyl. c is from 0 to about 4, preferably 0 or 1. Each R¹⁴ and R^{14′} is independently selected from hydrogen, alkyl, alkenyl, alkynyl, aryl, heteroalkyl, heteroaryl, cycloalkyl, heterocycloalkyl, halogen, haloalkyl, hydroxy, and alkoxy; preferably each R¹⁴ is hydrogen and each R^{14′} is independently hydrogen or lower alkyl. D is selected from a covalent bond, —O—, —SO_{d}—, —C(═O)—, —C(═O)N(R¹⁶)—, —N(R¹⁶)—, and —N(R16)C(═O)—; preferably D is —O—, —S—, SO₂—, —C(═O)N(R¹⁶)—, —N(R¹⁶)—, and —N(R¹⁶)C(═O)—; more preferably D is —O—. d is from 0 to 2. R¹⁶ is selected from hydrogen, alkyl, alkenyl, alkynyl, aryl, heteroaryl, heteroalkyl, heteroaryl, cycloalkyl, heterocycloalkyl, and haloalkyl; R¹⁶ is preferably lower alkyl or aryl. T is —(CR¹⁷R^{17′})ₑ—R¹⁸. e is from 0 to about 4, preferably 0 or 1. Each R¹⁷ and R^{17′} is independently selected from hydrogen, alkyl, alkenyl, alkynyl, aryl, heteroalkyl, heteroaryl, cycloalkyl, heterocycloalkyl, halogen, haloalkyl, hydroxy, alkoxy and aryloxy; preferably each R¹⁷ is hydrogen and each R^{17′} is independently hydrogen or lower alkyl. R¹⁸ is selected from hydrogen, alkyl, alkenyl, alkynyl, halogen, heteroalkyl, haloalkyl, aryl, heteroaryl, cycloalkyl, and heterocycloalkyl; preferably R¹⁸ is lower alkyl or aryl. Alternatively, R¹⁷ and R¹⁸, together with the atoms to which they are bonded, join to form an optionally substituted heterocyclic ring containing from 5 to 8 (preferably 5 or 6) atoms of which 1 to 3 (preferably 1 or 2) are heteroatoms. Alternatively, R¹⁵ and R¹⁸, together with the atoms to which they are bonded, join to form an optionally substituted heterocyclic ring containing from 5 to 8 (preferably 5 or 6) atoms of which 1 to 3 (preferably 1 or 2) are heteroatoms.

III. Compound Preparation:

The compounds of the invention can be prepared using a variety of procedures. The starting materials used in preparing the compounds of the invention are known, made by known methods, or are commercially available. Particularly preferred syntheses are described in the following general reaction schemes. (The R groups used to illustrate the reaction schemes do not necessarily correlate to the respective R groups used to describe the various aspects of the Formula I compounds. That is, for example, R¹ in Formula (I) does not represent the same moiety as R¹ here). Specific examples for making the compounds of the present invention are set forth in Section VII, below.

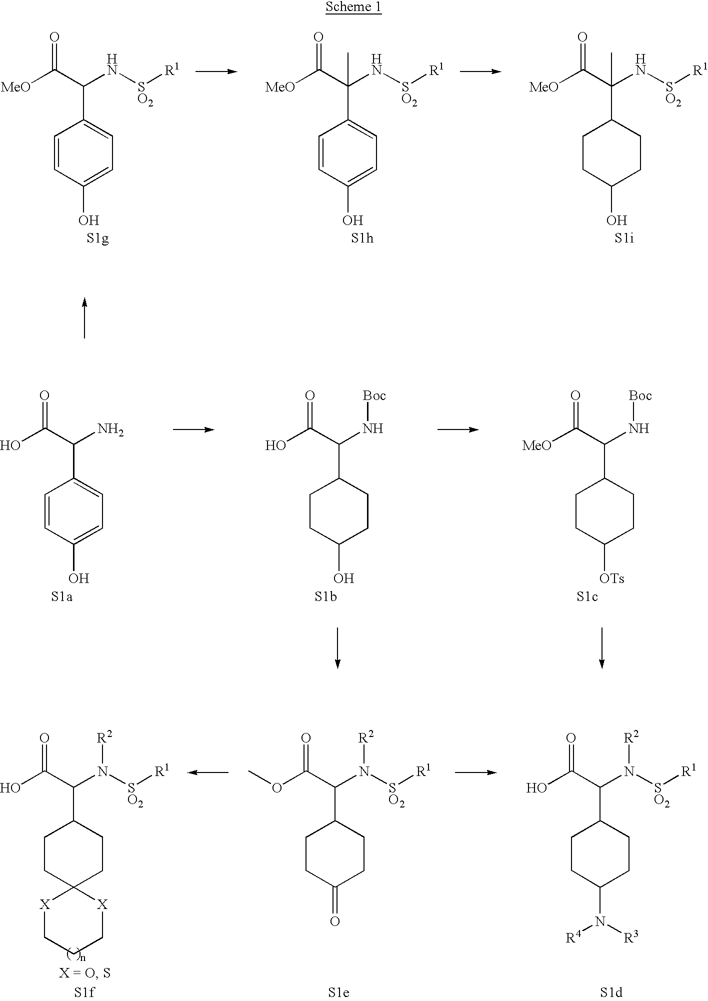

In Scheme 1, the aminoacid S1a is a commercially available material which is available in both enantiomeric forms. It can then be saturated under hydrogenation conditions to give S1b and then converted to tosylate S1c as described in the same manuscript. A sequence of well known transformations including displacement with sodium azide, hydrogenation to primary amine, amine functionalization and replacement of the boc protecting group with a sulfonyl chloride of choice then allows preparation of structures of type S1d. Alternatively, alcohol S1b could be converted to its relative sulfonamide and then oxidized to ketone S1e with Jones reagent. This then allows access to substituted amines of type S1d as well as spiroketals of type S1f.

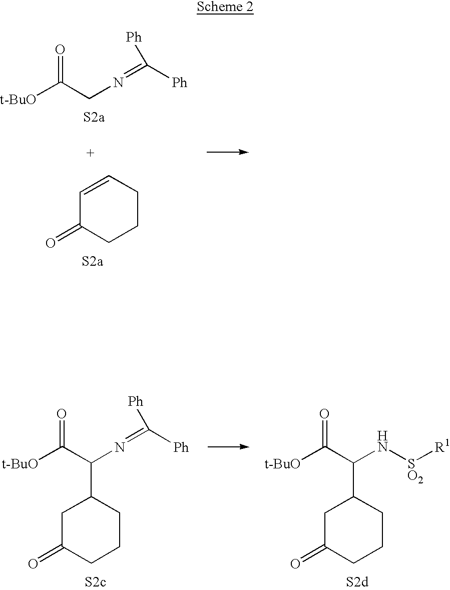

Enantioselective alkylation of S2a under phase transfer conditions is a well known method for the preparation of unnatural amino acids and the conjugate addition with enones such as cyclohexenone S2b to give ketones of type S2c as described by Corey et. al. Tetrahedron Lett. 1998, 5347. The imine S2c can then in turn be deprotected upon treatment with aqueous citric acid and sulfonylated with a sulfonyl chloride of choice to give ketone S2d, which can be functionalized as described in Scheme 1.

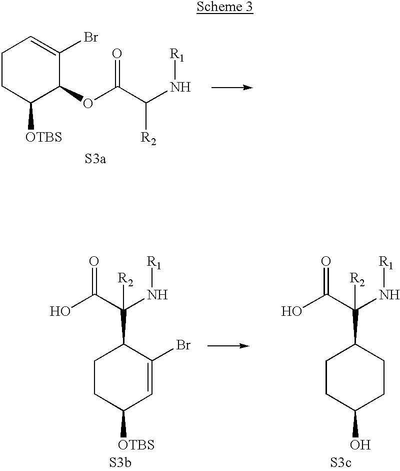

Esters of type S3a which are prepared from protected amino acids and allylic alcohols have been shown to undergo a Claisen rearrangement under strong base conditions to give entry to new amino acids of type S3b (Hudlicky, et. al J. Org. Chem. 1997, 62 1994). These can then in turn be manipulated as desired by the skilled artisan. One such manipulation is the reduction and deprotection of S3b to give S3c which provides an enantio- and diastereo-selective route to compounds of the type found in Scheme 2.

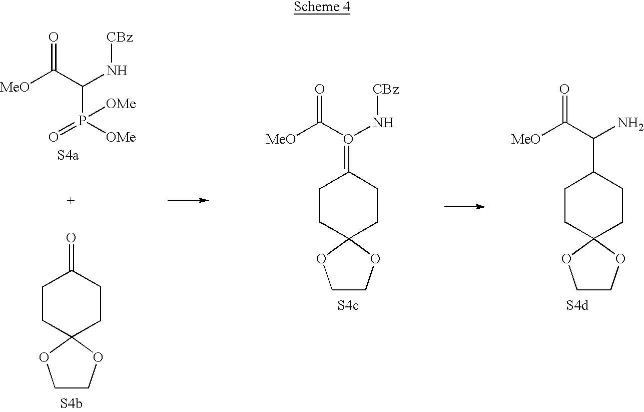

Esters of type S4c can be prepared under basic conditions by Wittig type coupling of commercially available substrates S4a and S4b. Catalytic hydrogenation then provides amino acids of type S4d. The free amine can then be sulfonylated using conditions well known in the art to give compounds of the type described in this invention. The ketal functionality can also be removed to reveal a ketone functionality which can be functionalized in many ways including those described in Scheme 1.

These steps may be varied to increase yield of desired product. The skilled artisan will recognize the judicious choice of reactants, solvents, and temperatures is an important component in any successful synthesis. Determination of optimal conditions, etc. is routine. Thus the skilled artisan can make a variety of compounds using the guidance of the schemes above.

It is recognized that the skilled artisan in the art of organic chemistry can readily carry out standard manipulations of organic compounds without further direction; that is, it is well within the scope and practice of the skilled artisan to carry out such manipulations. These include, but are not limited to, reduction of carbonyl compounds to their corresponding alcohols, oxidations of hydroxyls and the like, acylations, aromatic substitutions, both electrophilic and nucleophilic, etherifications, esterification and saponification and the like. Examples of these manipulations are discussed in standard texts such as March, Advanced Organic Chemistry (Wiley), Carey and Sundberg, Advanced Organic Chemistry (Vol. 2) and other art that the skilled artisan is aware of.

The skilled artisan will also readily appreciate that certain reactions are best carried out when another potentially reactive functionality on the molecule is masked or protected, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in T. Greene, Protecting Groups in Organic Synthesis. Of course, amino acids used as starting materials with reactive side chains are preferably blocked to prevent undesired side reactions.

The compounds of the invention may have one or more chiral centers. As a result, one may selectively prepare one optical isomer, including diastereomer and enantiomer, over another, for example by chiral starting materials, catalysts or solvents, or may prepare both stereoisomers or both optical isomers, including diastereomers and enantiomers at once (a racemic mixture). Since the compounds of the invention may exist as racemic mixtures, mixtures of optical isomers, including diastereomers and enantiomers, or stereoisomers may be separated using known methods, such as chiral salts, chiral chromatography and the like.

In addition, it is recognized that one optical isomer, including diastereomer and enantiomer, or stereoisomer may have favorable properties over the other. Thus when disclosing and claiming the invention, when one racemic mixture is disclosed, it is clearly contemplated that both optical isomers, including diastereomers and enantiomers, or stereoisomers substantially free of the other are disclosed and claimed as well.

IV. Methods of Use:

Metalloproteases (MPs) found in the body operate, in part, by breaking down the extracellular matrix, which comprises extracellular proteins and glycoproteins. Inhibitors of metalloproteases are useful in treating diseases caused, at least in part, by the breakdown of such proteins and glycoproteins. These proteins and glycoproteins play an important role in maintaining the size, shape, structure and stability of tissue in the body. Thus, MPs are intimately involved in tissue remodeling.

As a result of this activity, MPs have been said to be active in many disorders involving either the: (1) breakdown of tissues including opthalmic diseases; degenerative diseases, such as arthritis, multiple sclerosis and the like; and metastasis or mobility of tissues in the body; or (2) remodeling of tissues including cardiac disease, fibrotic disease, scarring, benign hyperplasia, and the like.

The compounds of the present invention prevent or treat disorders, diseases and/or unwanted conditions which are characterized by unwanted or elevated activity by MPs. For example, the compounds can be used to inhibit MPs which:

1. destroy structural proteins (i.e. the proteins that maintain tissue stability and structure);

2. interfere in inter/intracellular signaling, including those implicated in cytokine up-regulation, and/or cytokine processing and/or inflammation, tissue degradation and other maladies [Mohler K M, et al, Nature 370 (1994) 218-220, Gearing A J H, et al, Nature 370 (1994) 555-557 McGeehan G M, et al, Nature 370 (1994) 558-561]; and

3. facilitate processes which are undesired in the subject being treated, for example, the processes of sperm maturation, egg fertilization and the like.

As used herein, an “MP related disorder” or “MP related disease” is one that involves unwanted or elevated MP activity in the biological manifestation of the disease or disorder; in the biological cascade leading to the disorder; or as a symptom of the disorder. This “involvement” of the MP includes:

1. The unwanted or elevated MP activity as a “cause” of the disorder or biological manifestation, whether the activity is elevated genetically, by infection, by autoimmunity, trauma, biomechanical causes, lifestyle [e.g. obesity] or by some other cause;

2. The MP as part of the observable manifestation of the disease or disorder. That is, the disease or disorder is measurable in terms of the increased MP activity. From a clinical standpoint, unwanted or elevated MP levels indicate the disease, however, MPs need not be the “hallmark” of the disease or disorder; or

3. The unwanted or elevated MP activity is part of the biochemical or cellular cascade that results or relates to the disease or disorder. In this respect, inhibition of the MP activity interrupts the cascade, and thus controls the disease.

The term “treatment” is used herein to mean that, at a minimum, administration of a compound of the present invention mitigates a disease associated with unwanted or elevated MP activity in a mammalian subject, preferably in humans. Thus, the term “treatment” includes: preventing an MP-mediated disease from occurring in a mammal, particularly when the mammal is predisposed to acquiring the disease, but has not yet been diagnosed with the disease; inhibiting the MP-mediated disease; and/or alleviating or reversing the MP-mediated disease. Insofar as the methods of the present invention are directed to preventing disease states associated with unwanted MP activity, it is understood that the term “prevent” does not require that the disease state be completely thwarted. (See Webster's Ninth Collegiate Dictionary.) Rather, as used herein, the term preventing refers to the ability of the skilled artisan to identify a population that is susceptible to MP-related disorders, such that administration of the compounds of the present invention may occur prior to onset of the disease. The term does not imply that the disease state be completely avoided. For example, osteoarthritis (OA) is the most common rhueumatological disease with some joint changes radiologically detectable in 80% of people over 55 years of age. Fife, R. S., “A Short History of Osteoarthritis”, Osteoarthritis: Diagnosis and Medical/Surgical Management, R. W. Moskowitz, D. S. Howell, V. M. Goldberg and H. J. Mankin Eds., p 11-14 (1992). A common risk factor that increases the incidence of OA is traumatic injury of the joint. Surgical removal of the meniscus following knee injury increases the risk of radiographically detectable OA and this risk increases with time. Roos, H et al. “Knee Osteoarthritis After Menisectomy: Prevalence of Radiographic Changes After Twenty-one Years, Compared with Matched Controls.” Arthritis Rheum., Vol. 41, pp 687-693; Roos, H et al. “Osteoarthritis of the Knee After Injury to the Anterior Cruciate Ligament or Meniscus: The Influence of Time and Age.” Osteoarthritis Cartilege., Vol. 3, pp 261-267 (1995). Thus, this patient population is identifiable and could receive administration of a compound of the present invention before progression of the disease. Thus, progression of OA in such individuals would be “prevented”.

Advantageously, many MPs are not distributed evenly throughout the body. Thus, the distribution of MPs expressed in various tissues are often specific to those tissues. For example, the distribution of metalloproteases implicated in the breakdown of tissues in the joints is not the same as the distribution of metalloproteases found in other tissues. Though not essential for activity or efficacy, certain diseases, disorders, and unwanted conditions preferably are treated with compounds that act on specific MPs found in the affected tissues or regions of the body. For example, a compound which displays a higher degree of affinity and inhibition for an MP found in the joints (e.g. chondrocytes) would be preferred for treatment of a disease, disorder, or unwanted condition found there than other compounds which are less specific.

In addition, certain inhibitors are more bioavailable to certain tissues than others. Choosing an MP inhibitor which is more bioavailable to a certain tissue and which acts on the specific MPs found in that tissue, provides for specific treatment of the disease, disorder, or unwanted condition. For example, compounds of this invention vary in their ability to penetrate into the central nervous system. Thus, compounds may be selected to produce effects mediated through MPs found specifically outside the central nervous system.

Determination of the specificity of an inhibitor of a specific MP is within the skill of the artisan in that field. Appropriate assay conditions can be found in the literature. Specifically, assays are known for stromelysin and collagenase. For example, U.S. Pat. No. 4,743,587 references the procedure of Cawston, et al., Anal Biochem (1979) 99:340-345. See also, Knight, C. G. et al., “A Novel Coumarin-Labelled Peptide for Sensitive Continuous Assays of the Matrix Metalloproteases”, FEBS Letters, Vol. 296, pp. 263-266 (1992). The use of a synthetic substrate in an assay is described by Weingarten, H., et al., Biochem Biophy Res Comm (1984) 139:1184-1187. Any standard method for analyzing the breakdown of structural proteins by MPs can, of course, be used. The ability of compounds of the invention to inhibit metalloprotease activity can, of course, be tested in the assays found in the literature, or variations thereof. Isolated metalloprotease enzymes can be used to confirm the inhibiting activity of the invention compounds, or crude extracts which contain the range of enzymes capable of tissue breakdown can be used.

The compounds of this invention are also useful for prophylactic or acute treatment. They are administered in any way the skilled artisan in the fields of medicine or pharmacology would desire. It is immediately apparent to the skilled artisan that preferred routes of administration will depend upon the disease state being treated and the dosage form chosen. Preferred routes for systemic administration include administration perorally or parenterally.

However, the skilled artisan will readily appreciate the advantage of administering the MP inhibitor directly to the affected area for many diseases, disorders, or unwanted conditions. For example, it may be advantageous to administer MP inhibitors directly to the area of the disease, disorder, or unwanted condition such as in the area affected by surgical trauma (e.g., angioplasty), scarring, burning (e.g., topical to the skin), or for opthalmic and periodontal indications.

Because the remodeling of bone involves MPs, the compounds of the invention are useful in preventing prosthesis loosening. It is known in the art that over time prostheses loosen, become painful, and may result in further bone injury, thus demanding replacement. The need for replacement of such prostheses includes those such as in, joint replacements (for example hip, knee and shoulder replacements), dental prosthesis, including dentures, bridges and prosthesis secured to the maxilla and/or mandible.

MPs are also active in remodeling of the cardiovascular system (for example, in congestive heart failure). It has been suggested that one of the reasons angioplasty has a higher than expected long term failure rate (reclosure over time) is that MP activity is not desired or is elevated in response to what may be recognized by the body as “injury” to the basement membrane of the vessel. Thus regulation of MP activity in indications such, as dilated cardiomyopathy, congestive heart failure, atherosclerosis, plaque rupture, reperfusion injury, ischemia, chronic obstructive pulmonary disease, angioplasty restenosis and aortic aneurysm may increase long term success of any other treatment, or may be a treatment in itself.

In one aspect of the present invention, the compounds of Formula I of the present invention may be effective in preventing or treating myocardial infarction (hereinafter “MI”). MI, also known as a “heart attack” or “heart failure,” is a condition caused by partial or complete occlusion of one or more of the coronary arteries, usually due to rupture of an atherosclerotic plaque. The occlusion of the coronary artery results in cardiac ischemia. MMPs are implicated in artherosclerotic plaque rupture. See e.g., Galis, Z. S., et al., J. Clin. Invest. 1994;94:2493-503; Lee, R. T., et al., Arterioscler. Thromb. Vasc. Biol. 1996;16:1070-73; Schonbeck, U. et al., Circulation Research 1997; 81(3), 448-454. Libby, P. et al., Circ. 1995;91:2844-50.

In another aspect of the invention, the compounds of the present invention may be effective in preventing or treating progressive ventricular dilation after a MI, the major contributing factor to the development of post-MI chronic heart failure (hereinafter “CHF”). Thus, in yet still another aspect of the invention, the compounds of the present invention may be effective in preventing or treating the development of post-MI chronic heart failure.

It is widely recognized that important structural changes occur within the ventricular myocardium following MI that results in alterations in LV geometry and function. These structural alterations occur in the infarct itself, in the border zone of the MI, and in regions remote from the MI that collectively result in progressive ventricular dilation and pump dysfunction. The most notable feature of this remodeling process is the region of the original MI appears to enlarge with thinning of the ventricular myocardial wall. This type of remodeling following the initial injury and healing process from an MI has been termed “infarct expansion.” A significant body of work suggests that treatment of acute myocardial infarction with an MMP inhibitor will limit the unfavorable dilation of the heart that occurs early after such an event and therefore improve outcomes by preventing long-term sequelae, such as the development of chronic heart failure. See, e.g., Spinale, F. G. et al., Circulation Research 82:482-495 (1998); McElmurray, J. H. I. et al., J. Pharmacol. Exp. Ther. 291:799-811 (1999); Thomas, C. V. et al., Circulation 97:1708-1715 (1998); Spinale, F. G. et al. Circ. 102:1944-49 (2000); Peterson, J. T. et al., Cardiovasc. Res., 46(2):307-15 (2000); Rohde, L. E. et al., Circ., 99:3063-70 (1999); Lindsey, M. L. et al., Circ. 105:753-58 (2002); Brinsa, T. A. et al., J. Cardiac Failure, 7 Suppl. 2:24 (2001); Mukherjee, R. et al., J. Cardiac Failure; 7 Suppl 2:7 (2001).

A suitable MI cardiac pharmacological model is described in Mukherjee, R. et al., J. Cardiac Failure; 7 Suppl 2:7 (2001). Briefly, pigs are prepared for the induction of myocardial infarction by implantation of an occlusion device on the circumflex coronary artery, and radiopaque markers are placed in the region destined to be infarcted to measure infarct expansion (see below). Measurements of left ventricular (hereinafter “LV”) volumes and distances between marker beads are made prior to and at various times after the induction of MI induced by activating the occlusion device.

The effects of selective MMP inhibition may be studied in a pig model of MI induced by ligation of the circumflex coronary artery. Animals are assigned to one of the following treatment groups: (1) 1 or 10 mg/kg three times a day of a compound of Formula (I) by oral administration starting 3 days prior to myocardial infarction; (2) 10 mg/kg three times a day of said compound by oral administration starting 3 days after MI; (3) MI with no active treatment; or (4) no myocardial infarction or drug treatment. At 10 days post-MI, LV end-diastolic volume (hereinafter “LVEDV”) is measured by ventriculography. LVEDV is increased in all MI groups. An attenuated increase in LVEDV by a compound of Formula (I) indicates that the compound may be effective in the prevention or treatment of progressive ventricular dilation, and thus the subsequent development of CHF.

In skin care, MPs are implicated in the remodeling or “turnover” of skin. As a result, the regulation of MPs improves treatment of skin conditions including but not limited to, wrinkle repair, regulation and prevention and repair of ultraviolet induced skin damage. Such a treatment includes prophylactic treatment or treatment before the physiological manifestations are obvious. For example, the MP may be applied as a pre-exposure treatment to prevent ultaviolet damage and/or during or after exposure to prevent or minimize post-exposure damage. In addition, MPs are implicated in skin disorders and diseases related to abnormal tissues that result from abnormal turnover, which includes metalloprotease activity, such as epidermolysis bullosa, psoriasis, scleroderma and atopic dermatitis. The compounds of the invention are also useful for treating the consequences of “normal” injury to the skin including scarring or “contraction” of tissue, for example, following burns. MP inhibition is also useful in surgical procedures involving the skin for prevention of scarring, and promotion of normal tissue growth including in such applications as limb reattachment and refractory surgery (whether by laser or incision).

In addition, MPs are related to disorders involving irregular remodeling of other tissues, such as bone, for example, in otosclerosis and/or osteoporosis, or for specific organs, such as in liver cirrhosis and fibrotic lung disease. Similarly in diseases such as multiple sclerosis, MPs may be involved in the irregular modeling of blood brain barrier and/or myelin sheaths of nervous tissue. Thus regulating MP activity may be used as a strategy in treating, preventing, and controlling such diseases.

MPs are also thought to be involved in many infections, including cytomegalovirus [CMV]; retinitis; HIV, and the resulting syndrome, AIDS.

MPs may also be involved in extra vascularization where surrounding tissue needs to be broken down to allow new blood vessels such as in angiofibroma and hemangioma.

Since MPs break down the extracellular matrix, it is contemplated that inhibitors of these enzymes can be used as birth control agents, for example in preventing ovulation, in preventing penetration of the sperm into and through the extracellular milieu of the ovum, implantation of the fertilized ovum and in preventing sperm maturation.

In addition they are also contemplated to be useful in preventing or stopping premature labor and delivery.

Since MPs are implicated in the inflammatory response and in the processing of cytokines, the compounds are also useful as anti-inflammatories, for use in disease where inflammation is prevalent including, inflammatory bowel disease, Crohn's disease, ulcerative colitis, pancreatitis, diverticulitis, asthma or related lung disease, rheumatoid arthritis, gout and Reiter's Syndrome.

Where autoimmunity is the cause of the disorder, the immune response often triggers MP and cytokine activity. Regulation of MPs in treating such autoimmune disorders is a useful treatment strategy. Thus MP inhibitors can be used for treating disorders including, lupus erythmatosis, ankylosing spondylitis, and autoimmune keratitis. Sometimes the side effects of autoimmune therapy result in exacerbation of other conditions mediated by MPs, here MP inhibitor therapy is effective as well, for example, in autoimmune-therapy-induced fibrosis.

In addition, other fibrotic diseases lend themselves to this type of therapy, including pulmonary disease, bronchitis, emphysema, cystic fibrosis, acute respiratory distress syndrome (especially the acute phase response).

Where MPs are implicated in the undesired breakdown of tissue by exogenous agents, these can be treated with MP inhibitors. For example, they are effective as rattle snake bite antidote, as anti-vessicants, in treating allergic inflammation, septicemia and shock. In addition, they are useful as antiparasitics (e.g., in malaria) and antiinfectives. For example, they are thought to be useful in treating or preventing viral infection, including infection which would result in herpes, “cold” (e.g., rhinoviral infection), meningitis, hepatitis, HIV infection and AIDS.

MP inhibitors are also thought to be useful in treating Alzheimer's disease, amyotrophic lateral sclerosis (ALS), muscular dystrophy, complications resulting from or arising out of diabetes, especially those involving loss of tissue viability, coagulation, Graft vs. Host disease, leukemia, cachexia, anorexia, proteinuria, and perhaps regulation of hair growth.

For some diseases, conditions or disorders MP inhibition is contemplated to be a preferred method of treatment. Such diseases, conditions or disorders include, arthritis (including osteoarthritis and rheumatoid arthritis), cancer (especially the prevention or arrest of tumor growth and metastasis), ocular disorders (especially corneal ulceration, lack of corneal healing, macular degeneration, and pterygium), and gum disease (especially periodontal disease, and gingivitis)

Compounds preferred for, but not limited to, the treatment of arthritis (including osteoarthritis and rheumatoid arthritis) are those compounds that are selective for the matrix metalloproteases and the disintegrin metalloproteases.

Compounds preferred for, but not limited to, the treatment of cancer (especially the prevention or arrest of tumor growth and metastasis) are those compounds that preferentially inhibit gelatinases or type IV collagenases.

Compounds preferred for, but not limited to, the treatment of ocular disorders (especially corneal ulceration, lack of corneal healing, macular degeneration, and pterygium) are those compounds that broadly inhibit metalloproteases. Preferably these compounds are administered topically, more preferably as a drop or gel.

Compounds preferred for, but not limited to, the treatment of gum disease (especially periodontal disease, and gingivitis) are those compounds that preferentially inhibit collagenases.

V. Compositions:

The compositions of the invention comprise:

(a) a safe and effective amount of a compound of the invention; and

(b) a pharmaceutically-acceptable carrier.

As discussed above, numerous diseases are known to be mediated by excess or undesired metalloprotease activity. These include tumor metastasis, osteoarthritis, rheumatoid arthritis, skin inflammation, ulcerations, particularly of the cornea, reaction to infection, periodontitis and the like. Thus, the compounds of the invention are useful in therapy with regard to conditions involving this unwanted activity.

The invention compounds can therefore be formulated into pharmaceutical compositions for use in treatment or prophylaxis of these conditions. Standard pharmaceutical formulation techniques are used, such as those disclosed in Remington's Pharmaceutical Sciences, Mack Publishing Company, Easton, Pa., latest edition.

A “safe and effective amount” of a Formula (I) compound is an amount that is effective, to inhibit metalloproteases at the site(s) of activity, in an animal, preferably a mammal, more preferably a human subject, without undue adverse side effects (such as toxicity, irritation, or allergic response), commensurate with a reasonable benefit/risk ratio when used in the manner of this invention. The specific “safe and effective amount” will, obviously, vary with such factors as the particular condition being treated, the physical condition of the patient, the duration of treatment, the nature of concurrent therapy (if any), the specific dosage form to be used, the carrier employed, the solubility of the Formula (I) compound therein, and the dosage regimen desired for the composition.

In addition to the subject compound, the compositions of the subject invention contain a pharmaceutically-acceptable carrier. The term “pharmaceutically-acceptable carrier”, as used herein, means one or more compatible solid or liquid filler diluents or encapsulating substances which are suitable for administration to an animal, preferably a mammal, more preferably a human. The term “compatible”, as used herein, means that the components of the composition are capable of being commingled with the subject compound, and with each other, in a manner such that there is no interaction which would substantially reduce the pharmaceutical efficacy of the composition under ordinary use situations. Pharmaceutically-acceptable carriers must, of course, be of sufficiently high purity and sufficiently low toxicity to render them suitable for administration to the animal, preferably a mammal, more preferably a human being treated.

Some examples of substances which can serve as pharmaceutically-acceptable carriers or components thereof are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the Tweens®; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is basically determined by the way the compound is to be administered.

If the subject compound is to be injected, the preferred pharmaceutically-acceptable carrier is sterile, physiological saline, with blood-compatible suspending agent, the pH of which has been adjusted to about 7.4.

In particular, pharmaceutically-acceptable carriers for systemic administration include sugars, starches, cellulose and its derivatives, malt, gelatin, talc, calcium sulfate, vegetable oils, synthetic oils, polyols, alginic acid, phosphate buffer solutions, emulsifiers, isotonic saline, and pyrogen-free water. Preferred carriers for parenteral administration include propylene glycol, ethyl oleate, pyrrolidone, ethanol, and sesame oil. Preferably, the pharmaceutically-acceptable carrier, in compositions for parenteral administration, comprises at least about 90% by weight of the total composition.

The compositions of this invention are preferably provided in unit dosage form. As used herein, a “unit dosage form” is a composition of this invention containing an amount of a Formula (I) compound that is suitable for administration to an animal, preferably a mammal, more preferably a human subject, in a single dose, according to good medical practice. These compositions preferably contain from about 5 mg (milligrams) to about 1000 mg, more preferably from about 10 mg to about 500 mg, more preferably from about 10 mg to about 300 mg, of a Formula (I) compound.

The compositions of this invention may be in any of a variety of forms, suitable (for example) for oral, rectal, topical, nasal, ocular or parenteral administration. Depending upon the particular route of administration desired, a variety of pharmaceutically-acceptable carriers well-known in the art may be used. These include solid or liquid fillers, diluents, hydrotropes, surface-active agents, and encapsulating substances. Optional pharmaceutically-active materials may be included, which do not substantially interfere with the inhibitory activity of the Formula (I) compound. The amount of carrier employed in conjunction with the Formula (I) compound is sufficient to provide a practical quantity of material for administration per unit dose of the Formula (I) compound. Techniques and compositions for making dosage forms useful in the methods of this invention are described in the following references, all incorporated by reference herein: Modern Pharmaceutics, Chapters 9 and 10 (Banker & Rhodes, editors, 1979); Lieberman et al., Pharmaceutical Dosage Forms: Tablets (1981); and Ansel, Introduction to Pharmaceutical Dosage Forms 2d Edition (1976).

Various oral dosage forms can be used, including such solid forms as tablets, capsules, granules and bulk powders. These oral forms comprise a safe and effective amount, usually at least about 5%, and preferably from about 25% to about 50%, of the Formula (I) compound. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carrier suitable for the preparation of unit dosage forms for peroral administration are well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical for the purposes of the subject invention, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, Avicel″ RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit″ coatings, waxes and shellac.

Compositions of the subject invention may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

The compositions of this invention can also be administered topically to a subject, e.g., by the direct laying on or spreading of the composition on the epidermal or epithelial tissue of the subject, or transdermally via a “patch”. Such compositions include, for example, lotions, creams, solutions, gels and solids. These topical compositions preferably comprise a safe and effective amount, usually at least about 0.1%, and preferably from about 1% to about 5%, of the Formula (I) compound. Suitable carriers for topical administration preferably remain in place on the skin as a continuous film, and resist being removed by perspiration or immersion in water. Generally, the carrier is organic in nature and capable of having dispersed or dissolved therein the Formula (I) compound. The carrier may include pharmaceutically-acceptable emollients, emulsifiers, thickening agents, solvents and the like.

VI. Methods of Administration:

This invention also provides methods of treating or preventing disorders associated with excess or undesired metalloprotease activity in a human or other animal subject, by administering a safe and effective amount of a Formula (I) compound to said subject. As used herein, a “disorder associated with excess or undesired metalloprotease activity” is any disorder characterized by degradation of matrix proteins. The methods of the invention are useful in treating or preventing disorders described above.

Compositions of this invention can be administered topically or systemically. Systemic application includes any method of introducing Formula (I) compound into the tissues of the body, e.g., intra-articular (especially in treatment of rheumatoid arthritis), intrathecal, epidural, intramuscular, transdermal, intravenous, intraperitoneal, subcutaneous, sublingual, rectal, and oral administration. The Formula (I) compounds of the present invention are preferably administered orally.

The specific dosage of inhibitor to be administered, as well as the duration of treatment, and whether the treatment is topical or systemic are interdependent. The dosage and treatment regimen will also depend upon such factors as the specific Formula (I) compound used, the treatment indication, the ability of the Formula (I) compound to reach minimum inhibitory concentrations at the site of the metalloprotease to be inhibited, the personal attributes of the subject (such as weight), compliance with the treatment regimen, and the presence and severity of any side effects of the treatment.

Typically, for a human adult (weighing approximately 70 kilograms), from about 5 mg to about 3000 mg, more preferably from about 5 mg to about 1000 mg, more preferably from about 10 mg to about 100 mg, of Formula (I) compound are administered per day for systemic administration. It is understood that these dosage ranges are by way of example only, and that daily administration can be adjusted depending on the factors listed above.

A preferred method of administration for treatment of rheumatoid arthritis is oral or parenterally via intra-articular injection. As is known and practiced in the art, all formulations for parenteral administration must be sterile. For mammals, especially humans, (assuming an approximate body weight of 70 kilograms) individual doses of from about 10 mg to about 1000 mg are preferred.

A preferred method of systemic administration is oral. Individual doses of from about 10 mg to about 1000 mg, preferably from about 10 mg to about 300 mg are preferred.

Topical administration can be used to deliver the Formula (I) compound systemically, or to treat a subject locally. The amounts of Formula (I) compound to be topically administered depends upon such factors as skin sensitivity, type and location of the tissue to be treated, the composition and carrier (if any) to be administered, the particular Formula (I) compound to be administered, as well as the particular disorder to be treated and the extent to which systemic (as distinguished from local) effects are desired.

The inhibitors of the invention can be targeted to specific locations where the metalloprotease is accumulated by using targeting ligands. For example, to focus the inhibitors to metalloprotease contained in a tumor, the inhibitor is conjugated to an antibody or fragment thereof which is immunoreactive with a tumor marker as is generally understood in the preparation of immunotoxins in general. The targeting ligand can also be a ligand suitable for a receptor which is present on the tumor. Any targeting ligand which specifically reacts with a marker for the intended target tissue can be used. Methods for coupling the invention compound to the targeting ligand are well known and are similar to those described below for coupling to carrier. The conjugates are formulated and administered as described above.

For localized conditions, topical administration is preferred. For example, to treat ulcerated cornea, direct application to the affected eye may employ a formulation as eyedrops or aerosol. For corneal treatment, the compounds of the invention can also be formulated as gels, drops or ointments, or can be incorporated into collagen or a hydrophilic polymer shield. The materials can also be inserted as a contact lens or reservoir or as a subconjunctival formulation. For treatment of skin inflammation, the compound is applied locally and topically, in a gel, paste, salve or ointment. For treatment of oral diseases, the compound may be applied locally in a gel, paste, mouth wash, or implant. The mode of treatment thus reflects the nature of the condition and suitable formulations for any selected route are available in the art.

In all of the foregoing, of course, the compounds of the invention can be administered alone or as mixtures, and the compositions may further include additional drugs or excipients as appropriate for the indication.

Some of the compounds of the invention also inhibit bacterial metalloproteases. Some bacterial metalloproteases may be less dependent on the stereochemistry of the inhibitor, whereas substantial differences are found between diastereomers in their ability to inactivate the mammalian proteases. Thus, this pattern of activity can be used to distinguish between the mammalian and bacterial enzymes.

### VII. EXAMPLES—COMPOUND PREPARATION

The following abbreviations are used herein:

| | |
|---|---|
| MeOH: | methanol |
| EtOAc: | ethylacetate |
| Ph: | phenyl |
| DMF: | N,N-dimethylformamide |
| conc.: | concentrated |
| rt: | room temperature |
| THF: | tetrahydrofuran |
| Et₃N: | triethylamine |
| Et₂O: | diethylether |
| boc: | t-butyloxycarbonyl |
| DME: | dimethoxyethane |
| wrt.: | with respect to |
| HOH: | potassium hydroxide |
| dil.: | dilute |

The R groups used to illustrate the compound examples do not correlate to the respective R groups used to describe the various moieties of Formula (I). That is, for example, R¹ and R² used to describe Formula (I) in the Summary of the Invention section and Section II of the Detailed Description section do not represent the same moieties as R¹ and R² in this Section VII.

### Examples 1-25

The following substructure and table show the structure of compounds made according to the procedures described in Examples 1-25. In these compounds, with reference to Formula (I), A is cyclohexane, R¹ is —OH and n=O.

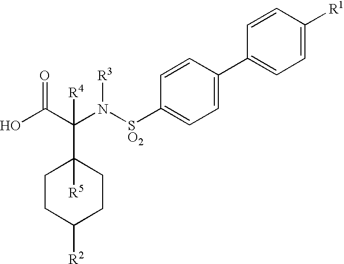

| Ex | R¹ | R² | R³ | R⁴ | R⁵ |
|---|---|---|---|---|---|
| 1 | —OMe | —OH | —Me | —H | —H |
| 2 | —OMe | 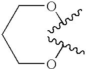 | —Me | —H | —H |
| 3 | —Br | 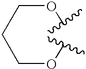 | —Me | —H | —H |
| 4 | —OMe | 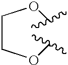 | —Me | —H | —H |
| 5 | —OMe | 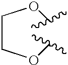 | —CH₂-4-Pyr | —H | —H |
| 6 | —OMe | 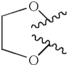 | —CH₂CH₂OMe | —H | —H |
| 7 | —OMe | 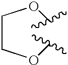 | —CH₂CH₂-N-morpholine | —H | —H |
| 8 | —OMe | 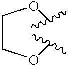 | —Me | —Me | —H |
| 9 | —OMe | 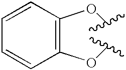 | —Me | —H | —H |
| 10 | —OMe | 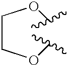 | —Me | —H | -SMe |
| 11 | —OMe | 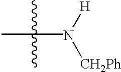 | —Me | —H | —H |
| 12 | —OMe | 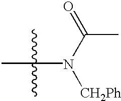 | —Me | —H | —H |
| 13 | —OMe | 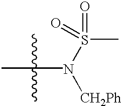 | —Me | —H | —H |
| 14 | —OMe | 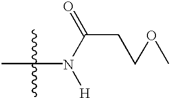 | —Me | —H | —H |
| 15 | —OMe | 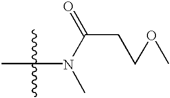 | —Me | —H | —H |
| 16 | —OMe | 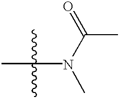 | —Me | —H | —H |
| 17 | —OMe | 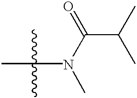 | —Me | —H | —H |
| 18 | —OMe | 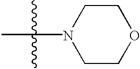 | —Me | —H | —H |
| 19 | —OMe | 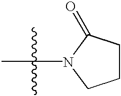 | —Me | —H | —H |
| 20 | —OMe | 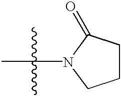 | —CH₂CH₂OMe | —H | —H |
| 21 | —OMe | 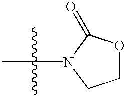 | —Me | —H | —H |
| 22 | —OMe | 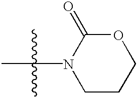 | —Me | —H | —H |
| 23 | —OMe | 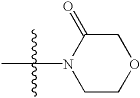 | —Me | —H | —H |
| 24 | —OMe | 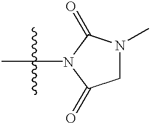 | —Me | —H | —H |
| 25 | —OMe | 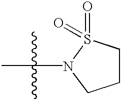 | —Me | —H | —H |

### Example 1

### Preparation of N-{[4′-Methoxy-(1,1′-biphenyl)-4-yl]-sulfonyl-N-methyl-amino}-(4-hydroxycyclohexan-1-yl)-acetic Acid

a. (R)-(4-Hydroxycyclohex-1-yl)-aminoacetic acid: The starting D-4-hydroxyphenyl glycene (10 g, 59.8 mmole) is taken in 180 mL of water in the presence of 10 mL of 50% NaOH and 25 g of Raney nickel. The mixture is pressurized to about 100 psi of hydrogen at 80° C. for 3 days, filtered through celite, and concentrated to about half of the original volume.

b. Methyl (R)-N-{[4′-Methoxy-(1,1′-biphenyl)-4-yl]-sulfonyl}-amino-(4-hydroxy-cyclohex-1-yl)-acetate: The crude amino acid 1a solution is diluted with 100 mL of dioxane and 10 mL of triethylamine and treated with [4′-methoxy-(1,1′-biphenyl)-4-yl]-sulfonyl chloride (18.6 g, 65.8 mmole). The resulting solution is stirred for 12 hr and then concentrated to about half of the original volume and acidified with con. HCl. The resulting white precipitate is washed with water and dried on a filter. This material is then taken in 150 mL of methanol, treated with 12 mL of thionyl chloride, stirred for 16 hr., and concentrated to dryness. The crude material is purified by chromatography with EtOAc to give the desired material as a white solid.

c. Methyl (R)-N-{[4′-Methoxy-(1,1′-biphenyl)-4-yl]-sulfonyl}-N-methylamino-(4-hydroxy-cyclohex-1-yl)-acetate: To a solution of sulfonamide 1b (1.15 g) in dimethylformamide (8 mL) is added anhydrous potassium carbonate (0.90 g) followed by methyl iodide (1.0 mL) and the reaction mixture is stirred at room temperature overnight. The solvents are removed under reduced pressure and the residue is diluted with methylene chloride and washed successively with water, brine, and then dried (Na₂SO₄). The crude product obtained after evaporation of solvents is purified using RP-HPLC to give the desired product as a colorless solid.

d. The ester 1b (245 mg, 0.56 mmole) is taken in 10 mL of methanol with 1 mL of water and treated with 300 mg of KOH. The resulting mixture is stirred for 16 hr and then concentrated to dryness. The residue is partitioned between EtOAc and 1N HCl. The organic layer is washed with brine, dried over MgSO₄, filtered and evaporated. The solid residue is recrystallized from EtOAc:hexanes to give the title acid as a white solid.

### Example 2

### Preparation of (R)-N-{[4′-methoxy-(1,1′-biphenyl)-4-yl]-sulfonyl}-N-methylamino-(1,5-dioxa-spiro[5.5]undec-9-yl)-acetic Acid

a. Methyl (R)-N-{[4′-Methoxy-(1,1′-biphenyl)-4-yl]-sulfonyl}-N-methylamino-(4-oxocyclohex-1-yl)-acetate: The starting alcohol 1c (4.1 g, 9.73 mmole) is taken in 200 mL of acetone and treated dropwise with Jones reagent (2.8 mL, 8 M, 25 mmole). The resulting solution is stirred for 3 hr and then quenched with 10 mL of isopropyl alcohol. The resulting slurry is filtered through a plug of silica with EtOAc to give the desired compound as a white solid.

b. Methyl (R)-N-{[4′-Methoxy-(1,1′-biphenyl)-4-yl]-sulfonyl}-N-methylamino-(1,5-dioxa-spiro[5.5]undec-9-yl)-acetate: The starting ketone 2a (328 mg, 0.77 mmole) is taken in 25 mL of benzene and treated with 1,3-propanediol (0.13 mL, 1.6 mmole) in the presence of catalytic para-toluenesulfonic acid and activated 4 Å molecular sieves. The mixture is refluxed for 16 hr, filtered through celite and evaporated. The residue is purified over flash silica with hexanes:EtOAc (1:1) to give a colorless oil.

c. The ester 2b is hydrolyzed as described for compound 1d to give the title acid.

### Example 3

### Preparation of (R)-N-{[4′-bromo-(1,1′-biphenyl)-4-yl]-sulfonyl}-N-methylamino-(1,5-dioxa-spiro[5.5]undec-9-yl)-acetic Acid

a. Methyl (R)-N-{[4′-bromo-(1,1′-biphenyl)-4-yl]-sulfonyl}-amino-(4-hydroxy-cyclohex-1-yl)-acetate: The starting glycene 1a is coupled with [4′-bromo-(1,1′-biphenyl)-4-yl]-sulfonyl chloride as described for compound 1b and then methylated as described for compound 1c.

b. The starting alcohol 3a is carried forward to the title acid as described by the sequence of reactions for compounds 2a-c.

### Example 4

### Preparation of (1,4-Dioxa-spiro[4.5]dec-8-yl)-N-{[4′-Methoxy-(1,1′-biphenyl)-4-yl]-sulfonyl}-N-methylamino-acetic Acid

a. N-Benzyloxycarbonylamino-(1,4-dioxa-spiro[4.5]dec-8-ylidene)-acetic acid methyl ester. To a solution of 1,4-dioxa-spiro[4.5]decan-8-one (1.56 g) and benzyloxycarbonylamino-(dimethoxy-phosphoryl)-acetic acid methyl ester (3.31 g) in dichloromethane (20 mL) cooled to 0° C. is added dropwise diazabicycloundecane (1.82 g). The resulting mixture is stirred at rt for 5 days. The solvent is removed under reduced pressure and the mixture is dissolved in EtOAc. The organic extracts are washed with water followed by brine, then dried (Na₂SO₄). The crude product obtained after evaporation of solvent is purified by chromatography on silica gel using 3/2 hexane/EtOAc to provide the desired product as a white solid.

b. Amino-(1,4-dioxa-spiro[4.5]dec-8-yl)-acetic acid methyl ester. The starting protected amine 4a (1.81 g) is dissolved in methanol (20 mL) and 10% palladium on carbon (200 mg) is added. The flask is flushed with hydrogen and the reaction mixture is stirred at room temperature for 12 hours. The reaction mixture is filtered through a Celite plug and the solvent is evaporated under reduced pressure to give the desired product which is used in the following reaction without purification.

c) Methyl (1,4-dioxa-spiro[4.5]dec-8-yl)-N-{[4′-Methoxy-(1,1′-biphenyl)-4-yl]-sulfonyl}-amino-acetate: To a solution of starting amine 4b (572 mg) in dichloromethane (10 mL) is added triethylamine (0.5 mL) followed by 4′-methoxy-biphenyl-4-sulfonyl chloride (850 mg). The reaction mixture is stirred overnight at room temperature, washed sequentially with 1N hydrochloric acid, water, 5% aqueous sodium bicarbonate and brine, then dried (Na₂SO₄). The crude product obtained after evaporation of solvent is purified by chromatography on silica gel using 3/2 hexane/EtOAc to provide the desired product as a colorless solid.

d) Methyl (1,4-dioxa-spiro[4.5]dec-8-yl)-N-{[4′-Methoxy-(1,1′-biphenyl)-4-yl]-sulfonyl}-N-methylamino-acetate: The starting sulfonamide 4c is methylated as described for compound 2c.

e) To a solution of ester 4c (390 mg, 080 mmole) in tetrahydrofuran (10 mL) is added 50% sodium hydroxide (1.0 mL) and the reaction mixture is stirred overnight at rt. The reaction mixture is concentrated under reduced pressure, diluted with EtOAc and washed successively with 1N HCl, water, brine, and then dried (Na₂SO₄). The crude product obtained after evaporation of solvent is purified by crystallization from methanol/water to give the title acid as a white solid.

### Example 5

### Preparation of (1,4-Dioxa-spiro[4.5]dec-8-yl)-N-{[4′-Methoxy-(1,1′-biphenyl)-4-yl]-sulfonyl}-N-(pyridin-4-yl-methyl)-amino-acetic Acid

a) Methyl (1,4-dioxa-spiro[4.5]dec-8-yl)-N-{[4′-Methoxy-(1,1′-biphenyl)-4-yl]-sulfonyl}-N-(pyridin-4-yl-methyl)-amino-acetate: The starting sulfonamide 4c is coupled with pyridin-4-yl-methyl bromide as described for compound 2c.

b) The starting ester 5a (382 mg, 0.68 mmole) is taken in 10 mL of methanol:water (10:1), treated with 600 mg of KOH, stirred for 3 days and evaporated to dryness. The residue is partitioned between EtOAc and 1N HCl. A solid forms at the interface of the two layers and the EtOAc portion is filtered through a Büchner funnel to give the title acid as a white solid.

### Example 6

### Preparation of (1,4-Dioxa-spiro[4.5]dec-8-yl)-N-{[4′-Methoxy-(1,1′-biphenyl)-4-yl]-sulfonyl}-N-(2-methoxyethyl)-amino-acetic Acid

The starting sulfonamide 4c is coupled with 2-methoxyethyl bromide as described for compound 2c and then hydrolyzed as described for compound 4e to give the title acid as a white solid.

### Example 7

### Preparation of (1,4-Dioxa-spiro[4.5]dec-8-yl)-N-{[4′-Methoxy-(1,1′-biphenyl)-4-yl]-sulfonyl}-N-(N-morpholinoethyl)-amino-acetic Acid

The starting sulfonamide 4c is coupled with N-morpholineoethyl bromide as described for compound 2c and then hydrolyzed as described for compound 5b to give the title acid. As a white solid.

### Example 8

### Preparation of 2-(1,4-Dioxa-spiro[4.5]dec-8-yl)-2N-{[4′-Methoxy-(1,1′-biphenyl)-4-yl]-sulfonyl}-methylamino-propionic Acid

a. Methyl 2-(1,4-dioxa-spiro[4.5]dec-8-yl)-2N-{[4′-Methoxy-(1,1′-biphenyl)-4-yl]-sulfonyl}-amino-propionate: Sulfonamide 4c (4.8 g, 10.1 mmole) is taken in 25 mL of THF, cooled to −78° C., and treated dropwise via cannula with a solution of lithium diisopropylamide (20 mL, 1.26 M in THF, 25.2 mmole). The resulting solution is stirred at −78° C. for 30 min., then warmed to −10° C. for 10 min., and recooled to −78° C. Methyl iodide (5.0 mL, 77.3 mmole) is added and the resulting solution is stirred for 1 hr and then warmed to −10° C. for 15 min. and quenched with saturated NH₄Cl. This mixture is then partitioned between water and EtOAc. Combined organic layers are then washed with brine and then dried over MgSO₄, filtered and evaporated. The crude material is purified by chromatography with hexanes:EtOAc to give the desired material.

b. Methyl 2-(1,4-Dioxa-spiro[4.5]dec-8-yl)-2N-{[4′-Methoxy-(1,1′-biphenyl)-4-yl]-sulfonyl}-methylamino-propionate: To a solution of sulfonamide 8a (1.38 g) in dimethylformamide (8 mL) is added anhydrous potassium carbonate (1.0 g) followed by methyl iodide (1.5 mL) and the reaction mixture is heated to 60° C. for 16 hr. The solvents are removed under reduced pressure and the residue is diluted with methylene chloride and washed successively with water, brine, and then dried (Na₂SO₄). The crude product obtained after evaporation of solvents is purified using RP-HPLC to give the desired product as a colorless solid.

c. The starting ester 8b (904 mg, 1.8 mmole) is taken in 10 mL of pyridine in the presence of Lithium Iodide (2.0 g, 15 mmole) and brought to reflux for 6 hr. The mixture is then diluted in EtOAc and washed 3 times with 1N HCl, 1 time with with brine, dried over MgSO₄, filtered and evaporated to give a crude solid which is recrystallized from hexanes:EtOAc.

### Example 9

### Preparation of [Spiro-(1,3-benzodioxole-2,1′-cyclohex-4′-yl]-N-{[4′-Methoxy-(1,1′-biphenyl)-4-yl]-sulfonyl}-N-methylamino-acetic Acid

The starting ketone 2a is condensed with 1,2-hydroxybenzene as described for compound 2b and then hydrolyzed as described for compound 1d.

### Example 10

### Preparation of (7-methylthio-1,4-Dioxaspiro[4.5]dec-7-yl)-N-{[4′-Methoxy-(1,1′-biphenyl)-4-yl]-sulfonyl}-N-methylamino-acetic Acid

a. Ethyl N-formyl- -(1,4-dioxaspiro[4.5]dec-7-yl)-ideneglycinate: A suspension of sodium hydide (4.07 g, 60%, 101 mmol) in THF (100) is cooled to 0° C. Two addition funnels are charged with ethyl isocyanoacetate (10.0 g, 88.4 mmol) in THF (10 mL) and 1,4-dioxa-spiro[4.5]decan-8-one (13.8 g, 88.4 mmol) in THF (10 mL), respectively. The solutions are added dropwise to the reaction mixture over a 30 minute period. The resulting mixture is then allowed to warm to rt and stirred overnight. The reaction mixture is quenched by the addition of saturated ammonium chloride solution. The layers are separated, and the aqueous layer is washed with EtOAc (3×100 mL). The combined organic extracts are washed with brine (200 mL), dried (MgSO₄) and then concentrated to an oil under reduced pressure. Ethyl acetate (40 mL) followed by hexane is added to the mixture until the mixture became turbid. The resulting solution is cooled to 0° C. and the desired product crystallized out of solution.

b. Ethyl N-formyl-amino-(7-methylthio-1,4-dioxaspiro[4.5]dec-8-yl)-acetate: The glycinate 10a (1.25 g, 4.74 mmol) in methanol (25 mL) is stirred at rt and then sodium thiomethoxide (0.66 g, 9.5 mmol, 2 equiv) is added. The resulting mixture is stirred at rt overnight. The reaction is quenched by the addition of saturated sodium bicarbonate solution. The resulting mixture is extracted with methylene chloride (3×100 mL). The organic extracts are dried over MgSO₄ and then concentrated to an oil under reduced pressure. The product is purified by chromatography on silica gel (7/3 EtOAC/hexane as eluent) to obtain the desired product as a clear colorless oil.

c. Amino-(7-methylthio-1,4-Dioxaspiro[4.5]dec-7-yl)-acetic acid: The formate ester 10b (0.75 g, 2.44 mmol) is stirred in 4 N HCl (50 mL) and heated to reflux overnight. The reaction mixture is then cooled to rt and then the solvent is removed under reduced pressure to leave the desired product as a white solid.

d. The amino acid, hydrochloric acid salt 10c (485 mg, 1.61 mmol) in THF (10 mL) and water (5 mL) is stirred at rt and then a solution of sodium bicarbonate (406 mg, 4.83 mmole) in 5 mL of water followed by p-methoxybiphenylsulfonyl chloride (683 mg, 2.41 mmol, 1.5 equiv) is added. The resulting mixture is stirred at rt overnight. The mixture is diluted with EtOAc and water, acidified with 1N HCl, and the aqueous layer is extracted with additional EtOAc. The organic extracts are rinsed with brine, dried (MgSO₄), and then concentrated to a solid residue under reduced pressure. The residue is purified by preparative HPLC, giving the product as a colorless solid.

### Example 11

### Preparation of N-{[4′-Methoxy-(1,1′-biphenyl)-4-yl]-sulfonyl-N-methylamino}-[4-(N-benzylamino)-cyclohexan-1-yl]-acetic Acid

a. Methyl N-{[4′-Methoxy-(1,1′-biphenyl)-4-yl]-sulfonyl}-N-methylamino-[4-oxo-cyclohex-1-yl]-acetate: The spiroketal 4d (10 g, 21.0 mmole) is taken in 150 mL of THF and treated with 150 mL of 1N HCl. The resulting mixture is stirred at rt for 18 hr and then diluted with 500 mL of EtOAc. The aqueous phase is back extracted and the combined organic layers are washed with brine, dried over MgSO₄, filtered and evaporated to give a solid which is purified over flash silica with hexanes:EtOAc (1:1) to give a white solid.

b. Methyl N-{[4′-Methoxy-(1,1′-biphenyl)-4-yl]-sulfonyl}-N-methylamino-[4-(N-benzyl-amino)-cyclohex-1-yl]-acetate: The ketone 11a (3.0 g, 6.9 mmole) is taken in 15 mL of methanol which is buffered with HOAc/NaOAc and treated with benzyl amine (0.7 mL, 6.4 mmole) and NaCNBH₃ (436 mg, 6.96 mmole). The resulting solution is stirred for 16 hr and then partitioned between 5% Na₂CO₃ and EtOAc. The organic layer is washed with brine, dried over MgSO₄, filtered and evaporated. The residue is purified over flash silica with EtOAc to give the desired compound as a 2:1 mixture of diastereomers.

c. The starting ester 11b (300 mg, 0.56 mmole) is taken in 10 ML of methanol:water (10:1), treated with KOH (600 mg, 10.4 mmole), stirred for two days, evaporated and partitioned between EtOAc and 1N HCl. A white solid is formed at the interface which is filtered and dried under vacuum to give the title acid as a white solid.

### Example 12

### Preparation of N-{[4′-Methoxy-(1,1′-biphenyl)-4-yl]-sulfonyl-N-methylamino}-[4-(N-benzyl-N-acetyamino)-cyclohexan-1-yl]-acetic Acid

a. Methyl N-{[4′-Methoxy-(1,1′-biphenyl)-4-yl]-sulfonyl}-N-methylamino-[4-(N-benzyl-N-acetylamino)-cyclohex-1-yl]-acetate: The starting benzyl amine 11b (330 mg, 0.62 mmole) is taken in 10 mL of CH₂Cl₂ in the presence of 2 mL of NEt₃ and treated with acetyl chloride (0.055 mL, 0.85 mmole) and the resulting solution is stirred for 3 hr and then partitioned between 1N HCl and EtOAc. The organic layer is washed with brine, dried over MgSO₄, filtered and evaporated to give a solid which is purified over flash silica with Hexanes:EtOAc to give a white solid.

b. The ester 12a is hydrolyzed as described for compound 4e.

### Example 13

### Preparation of N-{[4′-Methoxy-(1,1′-biphenyl)-4-yl]-sulfonyl-N-methylamino}-[4-(N-benzyl-N-methanesulfonylamino)-cyclohex-1-yl]-acetic Acid

The starting benzyl amine 11b is coupled with methanesulfonyl chloride and then hydrolyzed as described for compounds 12a-b.

### Example 14

### Preparation of N-{[4′-Methoxy-(1,1′-biphenyl)-4-yl]-sulfonyl-N-methylamino}-(4-N-methoxymethylacetylamino-cyclohexan-1-yl)-acetic Acid

a. N-{[4′-Methoxy-(1,1′-biphenyl)-4-yl]-sulfonyl-N-methylamino}-(4-N-amino-cyclohexan-1-yl)-acetic acid: The starting benzylamine 11b (3.34 g, 6.2 mmole) is taken in 50 mL of methanol in the presence of 200 mg of 10% palladium-hydroxide on carbon and shaken under 43 psi of hydrogen for 16 hr. The mixture is then purged with nitrogen, filtered through a pad of celite and evaporated to give a solid which is carried forward wothout purification.

b. The starting amine 14a is coupled with 3-methoxypropanyl chloride and hydrolyzed as described for compounds 12a-b.

### Example 15

### Preparation of N-{[4′-Methoxy-(1,1′-biphenyl)-4-yl]-sulfonyl-N-methylamino}-(4-N-methoxymethylacetyl-N-methylamino-cyclohexan-1-yl)-acetic Acid

a. Methyl N-{[4′-Methoxy-(1,1′-biphenyl)-4-yl]-sulfonyl-N-methylamino}-(4-N-methylamino-cyclohexan-1-yl)-acetate: The ketone 11a is condensed with methyl amine as described for compound 11b.

b. The methyl amine 15a is coupled to 3-methoxypropanyl chloride and hydrolyzed as described for compounds 12a-b.

### Example 16

### Preparation of N-{[4′-Methoxy-(1,1′-biphenyl)-4-yl]-sulfonylamino}-(4-N-acetyl-N-methylamino-cyclohexan-1-yl)-acetic Acid

The methylamine 15a is acylated and hydrolyzed as described for compounds 12a-b to give the title acid.

### Example 17

### Preparation of N-{[4′-Methoxy-(1,1′-biphenyl)-4-yl]-sulfonyl-N-methylamino}-(4-N-dimethylacetyl-N-methyl-aminocyclohexan-1-yl)-acetic Acid

The methylamine 15a is acylated and hydrolyzed as described for compounds 12a-b to give the title acid.

### Example 18

### Preparation of N-{[4′-Methoxy-(1,1′-biphenyl)-4-yl]-sulfonyl-N-methylamino}-[4-(morpholin-4N-yl)-cyclohexan-1-yl]-acetic Acid

a. Methyl N-{[4′-Methoxy-(1,1′-biphenyl)-4-yl]-sulfonyl-N-methylamino}-[4-(morpholin-4N-yl)-cyclohexan-1-yl]-acetate: The free amine 14a (534 mg, 1.17 mmole) is taken in 5 mL of dimethylformamide in the presence of 1 mL of Et₃N, treated with bromoethyl ether (0.2 mL, 1.6 mmole) and heated to 60° C. for 16 hr. The resulting solution is then diluted with EtOAc, washed three times with 5% Na₂CO₃, one time with brine, dried over MgSO₄, filtered and evaporated. The residue is purified over flash silica with EtOAc:MeOH (10:0 to 10:1) to give a solid.

b. The morpholine 15a (317 mg, 0.61 mmole) is taken in 3 mL of MeOH:THF (1:1), treated with 5 drops of 50% NaOH, stirred for three hours and concentrated to dryness. The residue is taken in water and filtered through a plug of reverse phase silica first with water and then with water:CH₃CN (1:1). The water:CH₃CN fraction is evaporated to dryness to give the title acid as a white solid.

### Example 19

### Preparation of N-{[4′-Methoxy-(1,1′-biphenyl)-4-yl]-sulfonyl-N-methylamino}-[4-(2-oxopyrrolidin-1N-yl)-cyclohexan-1-yl]-acetic Acid

a. Methyl N-{[4′-Methoxy-(1,1′-biphenyl)-4-yl]-sulfonyl-N-methylamino}-[4-(2-oxopyrrolidin-1N-yl)-cyclohexan-1-yl]-acetate: The free amine 14a (748 mg, 1.67 mmole) is taken in 10 mL of dimethylformamide in the presence of 2 mL of Et₃N, treated with 4-bromobutanyl chloride (0.35 mL, 3.0 mmole) and heated to 60° C. for 16 hr. The resulting solution is then diluted with EtOAc, washed with 1N HCl and brine, dried over MgSO₄, filtered and evaporated. The residue is purified over flash silica with hexanes:EtOAc (1:1 to 1:9) to give a solid.

b. The lactam 19a is hydrolyzed as described for compound 4d to give the title acid as a white solid.

### Example 20

### Preparation of N-{[4′-Methoxy-(1,1′-biphenyl)-4-yl]-sulfonyl-N-(2-methoxyethyl)-amino}-[4-(2-oxopyrrolidin-1N-yl)-cyclohexan-1-yl]-acetic Acid

The spiroketal 6a is carried forward to the title acid as described for compound 19b.

### Example 21

### Preparation of N-{[4′-Methoxy-(1,1′-biphenyl)-4-yl]-sulfonyl-N-methylamino}-[4-(oxazolidin-2-one-3N-yl)-cyclohexan-1-yl]-acetic Acid

a. Methyl N-{[4′-Methoxy-(1,1′-biphenyl)-4-yl]-sulfonyl-N-methylamino}-[(2-hydroxyethyl)-aminocyclohexan-1-yl]-acetate: The ketone 11a is condensed with ethanolamine as described for compound 11b.

b. Methyl N-{[4′-Methoxy-(1,1′-biphenyl)-4-yl]-sulfonyl-N-methylamino}-[4-(oxazolidin-2-one-3N-yl)-cyclohexan-1-yl]-acetate: The hydroxylamine 21a (800 g, 1.6 mmole) is taken in 10 mL of toluene in the presence of 1.5 mL of Et₃N, treated with carbonyldiimidazole (325 g, 2.0 mmole) and stirred for 16 hr at rt. The mixture is then taken in EtOAc, washed with 1N HCl, washed with brine, dried over MgSO₄, filtered and evaporated. The mixture is then chromatographed through flash silica with EtOAc to give the desired material as a separable mixture of isomers.

c. The ester 21b is hydrolyzed as described for compound 4d to give the title acid as a white solid.

### Example 22

### Preparation of N-{[4′-Methoxy-(1,1′-biphenyl)-4-yl]-sulfonyl-N-methylamino}-[4-([1,3]-oxazinan-2-one-3N-yl)-cyclohexan-1-yl]-acetic Acid

The ketone 11a is condensed with 3-propanolamine as described for compound 11b and then carried forward to the title acid as described for compounds 21b-c.

### Example 23

### Preparation of N-{[4′-Methoxy-(1,1′-biphenyl)-4-yl]-sulfonyl-N-methylamino}-[4-(2-oxomorpholin-1N-yl)-cyclohexan-1-yl]-acetic Acid

The amino alcohol 21a is coupled to bromoacetyl chloride and closed as described for compound 19a. Hydrolysis as described for compound 4d then delivers the title acid.

### Example 24

### Preparation of N-{[4′-Methoxy-(1,1′-biphenyl)-4-yl]-sulfonyl-N-methylamino}-[4-(3N-methyl-hydantoin-1N-yl)-cyclohexan-1-yl]-acetic Acid

a. Methyl N-{[4′-Methoxy-(1,1′-biphenyl)-4-yl]-sulfonyl-N-methylamino}-[4-(N-boc-N-methylamino-acetyl)-aminocyclohexan-1-yl]-acetate: The amine 14a (1.3 mg, 3.0 mmole) is taken in 5 mL of CH₂Cl₂:dioxane (1:1) in the presence of N-boc-sarcosine (0.95 g, 5.0 mmole) and 50 mg of 4-dimethylaminopyridine and treated with dicyclohexyl-carbodiimide (1.24 g, 5.0 mmole). The resulting solution is stirred for 16 hr, diluted with EtOAc, washed dil. NaHCO3, washed with brine, dried over MgSO₄, filtered and evaporated. The crude material is chromatographed over flash silica with EtOAc to give the desired material.

b. Methyl N-{[4′-Methoxy-(1,1′-biphenyl)-4-yl]-sulfonyl-N-methylamino}-[4-(3N-methyl-hydantoin-1N-yl)-cyclohexan-1-yl]-acetate: The amine 24a (1.2 g, 1.9 mmole) is taken in 20 mL of CH₂Cl₂ and treated with 3 mL of trifluoroacetate. The resulting solution is stirred for two hr and evaporated to dryness. The residue is taken in 20 mL of CH₂Cl₂ in the presence of 3 mL of Et₃N and treated with carbonyldiimidazole (827 mg, 5.1 mmole). The resulting solution is stirred at rt for 16 hr and then diluted with EtOAc, washed with 1N HCl, washed with brine, dried over MgSO₄, filtered and evaporated. The residue is chromatographed over flash silica with EtOAc to give the desired material.

c. The hydantoin 24b is hydrolyzed as described for compound 4d to give the title acid as a white solid.

### Example 25

### Preparation of N-{[4′-Methoxy-(1,1′-biphenyl)-4-yl]-sulfonyl-N-methylamino}-[4-(-sultam-1N-yl)-cyclohexan-1-yl]-acetic Acid

The starting amine 11a is coupled to 3-bromopropanesulfonyl chloride as described for compound 19a and then hydrolyzed as described for compound 4d.

### Examples 26-44

The following substructure and table show the structure of compounds made according to the procedures described in Examples 26-44. In these compounds, with reference to Formula (I), A is cyclohexane, R¹ is —OH and n=0.

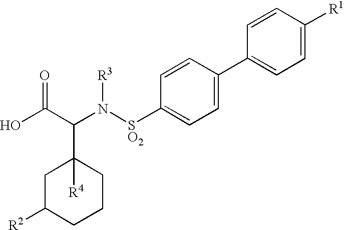

| Ex | R¹ | R² | R³ | R⁴ |
|---|---|---|---|---|
| 26 | —OMe | —OH | —Me | —H |
| 27 | —OMe | 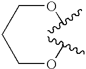 | —Me | —H |
| 28 | —Br | 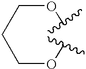 | —Me | —H |
| 29 | —OMe | 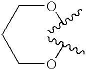 | —CH₂Ph | —H |
| 30 | —OMe | 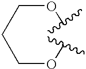 | —Me | —Me |
| 31 | —OMe | 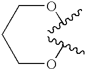 | —CH₂Ph | —Me |
| 32 | —OMe | —OCH₂Ph | —Me | —H |
| 33 | —OMe | 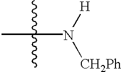 | —Me | —H |
| 34 | —OMe | 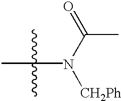 | —Me | —H |
| 35 | —OMe | 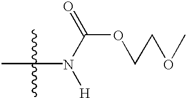 | —Me | —H |
| 36 | —OMe | 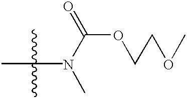 | —Me | —H |
| 37 | —OMe | 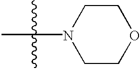 | —Me | —H |
| 38 | —OMe | 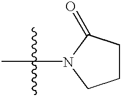 | —Me | —H |
| 39 | —OMe | 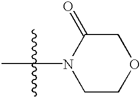 | —Me | —H |
| 40 | —OMe | 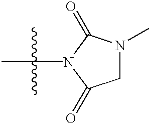 | —Me | —H |
| 41 | —OMe | 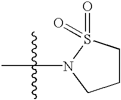 | —Me | —H |
| 42 | —OMe | 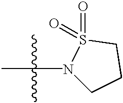 | —Me | —Me |
| 43 | —OMe | 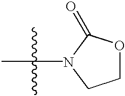 | —Me | —H |
| 44 | —OMe | 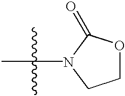 | —Me | —Me |

### Example 26

### Preparation of N-{[4′-methoxy-(1,1′-biphenyl)-4-yl]-sulfonyl}-N-methyl-amino-(3-hydroxycyclohexan-1-yl)-acetic Acid

a. Methyl glycinate benzophenone: The starting glycine methyl ester hydrochloride (20.2 g, 161 mmole) is taken in 250 mL of CH₂Cl₂ at rt under N₂ and treated with benzophenone imine (29.2 g, 161 mmole). The resulting heterogeneous mixture is vigorously stirred overnight and then filtered through a glass frit to remove ammonium salts. The filtrate is evaporated to dryness to give the desired product as a yellow oil which crystallizes at 0° C. No further purification is necessary. This type of transformation may also be performed asymmetrically (Tetrahedron Letters 1998, 39, 5347-5350, and references therein) to provide either enantiomer of 1a in enantiomerically-pure form.

b. Methyl (3-oxycyclohexan-1-yl)-glycinate benzophenone: To a stirred solution of diisopropylamine (13.1 g, 130 mmole) in 150 mL of THF at −78° C. under N₂ is added n-butyl lithium (12.4 mL, 10 M in hexanes). The solution is stirred for 45 min. and then methyl glycinate benzophenone 26a (30.0 g, 118 mmole) in 100 mL of THF is added dropwise. After an additional 45 min. cyclohexanone (11.3 g, 180 mmole) is added dropwise and the resulting solution is stirred for an additional 3 hr. The reaction is quenched at −78° C. with H₂O and allowed to warm to rt. The solution is further diluted with H₂O and extracted with CH₂Cl₂ (3×). The combined organic extracts are washed with brine, dried over MgSO₄, and evaporated to dryness to give the crude product a viscous orange oil. Purification by flash chromatography with 10%-20% EtOAc:hexanes provides the desired pure product as a mixture of diastereomers.

c. Methyl N-{[4′-methoxy-(1,1′-biphenyl)-4-yl]-sulfonyl}-amino-(3-oxycyclohexan-1-yl)-acetate: Following a literature procedure (Tetrahedron Letters 1997, 38 (49), 8595-8598), methyl (3-oxycyclohexan-1-yl)-glycinate benzophenone 26b (6.04 g, 17.3 mmole) is reacted with citric acid (20 mL, 15% wt/vol aqueous solution) in THF (40 mL) at rt for 5 hr. The solution is then extracted with Et₂O (2×) to remove byproduct benzophenone and any remaining starting material. The remaining aqueous solution is diluted with H₂O (30 mL) and the crude ammonium citrate is used without further purification. To this solution is added NaHCO₃ (approx. 20 g, excess) in portions. After the solution is completely neutralized and an excess of NaHCO₃ persists, the solution is diluted with dioxane (50 mL) and [4′-methoxy-(1,1′-biphenyl)-4-yl]-sulfonyl chloride (9.78 g, 34.6 mmole) is added. The slurry is then vigorously stirred overnight at rt. Afterwards, the solution is diluted with H₂O (500 mL) and extracted with CH₂Cl₂ (3×). The combined organic extracts are washed with brine, dried over MgSO₄ and evaporated to dryness to give the crude product as a white foam. Purification by flash chromatography with 25%-75% EtOAc: hexanes provides the desired product as an inseparable mixture of cis and trans diastereomers.

d. Methyl N-{[4′-methoxy-(1,1′-biphenyl)-4-yl]-sulfonyl}-N-methylamino-(3-oxycyclohexan-1-yl)-acetate: The sulfonamide 26c is methylated as described for compound 1c to give the title compound as a white solid.

e. Methyl N-{[4′-methoxy-(1,1′-biphenyl)-4-yl]-sulfonyl}-amino-(3-hydroxycyclo-hexan-1-yl)-acetate: To a stirred solution of ketone 26d (500 mg, 1.14 mmole) in MeOH:CH₂Cl₂ (3:1, 10 mL) at 0° C. under N₂ is added NaBH₄ (526 mg, 4.6 mmole). After 1 hr, the solution is diluted with H₂O (30 mL) and extracted with EtOAc (3×). The organic extracts are washed with brine, dried over MgSO₄ and evaporated to dryness to give the crude diastereomeric mixture as a white solid which requires no further purification.

f. Methyl ester 26e is hydrolyzed as described for compound 1d.

### Example 27

### Preparation of N-{[4′-methoxy-(1,1′-biphenyl)-4-yl]-sulfonyl}-N-methylamino-(1,5-dioxa-spiro[5.5]undec-7-yl)-acetic Acid

a. Methyl N-{[4′-methoxy-(1,1′-biphenyl)-4-yl]-sulfonyl}-N-methylamino-(1,5-dioxa-spiro[5.5]undec-7-yl)-acetate: Ketone 26d is reacted with 1,3-propanediol as described for compound 2b.

b. Methyl ester 26a is hydrolyzed as described for compound 4e to give the title acid.

### Example 28

### Preparation of N-{[4′-bromo-(1,1′-biphenyl)-4-yl]-sulfonyl}-N-methyl-amino-(1,5-dioxa-spiro[5.5]undec-7-yl)-acetic Acid

a. Methyl N-{[4′-bromo-(1,1′-biphenyl)-4-yl]-sulfonyl}-amino-(3-oxycyclohexan-1-yl)-acetate: Benzophenone imine 26b is hydrolyzed as described for compound 26c to give the intermediate ammonium citrate, which is coupled with [4′-bromo-(1,1′-biphenyl)-4-yl]-sulfonyl chloride as described for compound 24c.

b. Ketone 28a is carried forward to the title acid as described for compound 26d-f.

### Example 29

### Preparation of N-{[4′-methoxy-(1,1′-biphenyl)-4-yl]-sulfonyl}-N-benzylamino-(1,5-dioxa-spiro[5.5]undec-7-yl)-acetic Acid

The ketone 26c is benzylated as described for compound 1c and then carried forward to the title acid as described for compound 26e-f.

### Example 30

### Preparation of N-{[4′-methoxy-(1,1′-biphenyl)-4-yl]-sulfonyl}-N-methylamino-(7-methyl-1,5-dioxa-spiro[5.5]undec-7-yl)-acetic Acid

a. Methyl (1-methyl-3-oxycyclohexan-1-yl)-glycinate benzophenone: Imine 26a is coupled to 3-methyl-cyclohex-2-en-1-one as described for compound 26b.

b. Methyl N-{[4′-methoxy-(1,1′-biphenyl)-4-yl]-sulfonyl}-amino-(1-methyl-3-oxy-cyclohexan-1-yl)-acetate: The imine 30a is hydrolyzed and coupled to[4′-methoxy-(1,1′-biphenyl)-4-yl]-sulfonyl chloride as described for compound 26c.

c. Methyl N-{[4′-methoxy-(1,1′-biphenyl)-4-yl]-sulfonyl}-amino-(1-methyl-3-oxy-cyclohexan-1-yl)-acetate: Ketone 30b is methylated as described for compound 26d.

d. Ketone 30c is carried forward to the title acid as described for compounds 27a-b.

### Example 31

### Preparation of N-{[4′-methoxy-(1,1′-biphenyl)-4-yl]-sulfonyl}-N-benzylamino-(7-methyl-1,5-dioxa-spiro[5.5]undec-7-yl)-acetic Acid

Ketone 30b is benzylated as described for compound 26d and carried forward to the title acid as described for compounds 27a-b.

### Example 32

### Preparation of N-{[4′-methoxy-(1,1′-biphenyl)-4-yl]-sulfonyl}-N-methyl-amino-(3-benzyloxycyclohexan-1-yl)-acetic Acid

a. Methyl N-{[4′-methoxy-(1,1′-biphenyl)-4-yl]-sulfonyl}-amino-(3-benzyloxy-cyclohexan-1-yl)-acetate: Alcohol 26e (203 mg, 0.47 mmole) is taken in 15 mL of DMF and treated with sodium hydride (21 mg, 0.51 mmole, 60% dispersion in mineral oil). The resulting mixture is allowed to stir for 40 min. and then benzyl bromide is added (0.98 mL, 1.4 mmole). The mixture is stirred for 3 hr. and then diluted with 75 mL of water and extracted with ether. Combined ether layers are then washed with brine, dried over MgSO₄, filtered and evaporated. The crude material is is purified over flash silica with hexanes:EtOAc (2:1 to 1:2) to give two daistereomers of the desired material.

b. Ester 32a is hydrolyzed as described for compound 1d to give the title acid.

### Example 33

### Preparation of N-{[4′-Methoxy-(1,1′-biphenyl)-4-yl]-sulfonyl-N-methylamino}-[3-(N-benzylamino)-cyclohexan-1-yl]-acetic Acid

a. Methyl N-{[4′-Methoxy-(1,1′-biphenyl)-4-yl]-sulfonyl}-N-methylamino-[3-(N-benzyl-amino)-cyclohex-1-yl]-acetate: The ketone 26d is coupled to benzylamine as described for compound 11b.

b. Ester 32a is hydrolyzed as described for compound 11c.

### Example 34

### Preparation of N-{[4′-Methoxy-(1,1′-biphenyl)-4-yl]-sulfonyl-N-methylamino}-[3-(N-benzyl-N-acetyamino)-cyclohexan-1-yl]-acetic Acid

The benzylamine 33a is acetylated and hydrolyzed as described for compound 12a-b.

### Example 35

### Preparation of N-{[4′-Methoxy-(1,1′-biphenyl)-4-yl]-sulfonyl-N-methylamino}-(3-N-(2-methoxyethoxycarbonyl)-amino-cyclohexan-1-yl)-acetic Acid

a. Methyl N-{[4′-Methoxy-(1,1′-biphenyl)-4-yl]-sulfonyl-N-methylamino}-(3-amino-cyclohexan-1-yl)-acetate: The starting benzylamine 33a is hydrogenated as described for compound 14a to give a white solid.

b. Methyl N-{[4′-Methoxy-(1,1′-biphenyl)-4-yl]-sulfonyl-N-methylamino}-(3-N-(2-methoxyethoxycarbonyl)-amino-cyclohexan-1-yl)-acetate: The starting amine 35a is coupled to chloroformic acid 2-methoxyethyl ether as described for compound 14b to give a white solid.

c. The ester 35b is hydrolyzed as described for compound 4e.

### Example 36

### Preparation of N-{[4′-Methoxy-(1,1′-biphenyl)-4-yl]-sulfonyl-N-methylamino}-(3-N-methyl-N-(2-methoxyethoxycarbonyl)-amino-cyclohexan-1-yl)-acetic Acid

The free amine 35a is carried forward to the title acid as described for compound 15a-b.

### Example 37

### Preparation of N-{[4′-Methoxy-(1,1′-biphenyl)-4-yl]-sulfonyl-N-methylamino}-[3-(morpholin-4N-yl)-cyclohexan-1-yl]-acetic Acid

The free amine 35a is carried forward to the title acid as described for compound 18a-b.

### Example 38

### Preparation of N-{[4′-Methoxy-(1,1′-biphenyl)-4-yl]-sulfonyl-N-methylamino}-[3-(2-oxopyrrolidin-1N-yl)-cyclohexan-1-yl]-acetic Acid

The free amine 35a is carried forward to the title acid as described for compound 19a-b.

### Example 39

### Preparation of N-{[4′-Methoxy-(1,1′-biphenyl)-4-yl]-sulfonyl-N-methylamino}-[3-(2-oxomorpholin-1N-yl)-cyclohexan-1-yl]-acetic Acid

The free amine 35a is carried forward to the title acid as described for compound 23.

### Example 40

### Preparation of N-{[4′-Methoxy-(1,1′-biphenyl)-4-yl]-sulfonyl-N-methylamino}-[3-(3N-methyl-hydantoin-1N-yl)-cyclohexan-1-yl]-acetic Acid

The free amine 35a is carried forward to the title acid as described for compound 24a-c.

### Example 41

### Preparation of N-{[4′-Methoxy-(1,1′-biphenyl)-4-yl]-sulfonyl-N-methylamino}-[3-(□-sultam-1N-yl)-cyclohexan-1-yl]-acetic Acid

The free amine 35a is carried forward to the title acid as described for compound 25.

### Example 42

### Preparation of N-{[4′-Methoxy-(1,1′-biphenyl)-4-yl]-sulfonyl-N-methylamino}-[3-(□-sultam-1N-yl)-cyclohexan-1-yl]-acetic Acid

a. Methyl N-{[4′-Methoxy-(1,1′-biphenyl)-4-yl]-sulfonyl}-N-methylamino-[1-methyl-3-(N-benzyl-amino)-cyclohex-1-yl]-acetate: The ketone 30c is coupled to benzylamine as described for compound 11b.

b. Methyl N-{[4′-Methoxy-(1,1′-biphenyl)-4-yl]-sulfonyl-N-methylamino}-(1-methyl-3-amino-cyclohexan-1-yl)-acetate: The starting benzylamine 42a is hydrogenated as described for compound 14a to give a white solid.

c. The free amine 42b is carried forward to the title acid as described for compound 25.

### Example 43

### Preparation of N-{[4′-Methoxy-(1,1′-biphenyl)-4-yl]-sulfonyl-N-methylamino}-[3-(oxazolidin-2-one-3N-yl)-cyclohexan-1-yl]-acetic Acid

The ketone 26d is carried forward to the title acid as described for compound 21a-c.

### Example 44

### Preparation of N-{[4′-Methoxy-(1,1′-biphenyl)-4-yl]-sulfonyl-N-methylamino}-[1-methyl 3-(oxazolidin-2-one-3N-yl)-cyclohexan-1-yl]-acetic Acid

The ketone 30c is carried forward to the title acid as described for compound 21a-c.

### Examples 45 and 46

The following substructure and table show the structure of compounds made according to the procedures described in Examples 45 and 46. In these compounds, with reference to Formula (I), A is cyclopentane, R¹ is —OH and n=0.

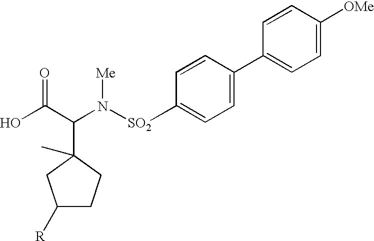

| Example | R |
|---|---|
| 45 | 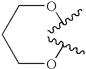 |
| 46 | 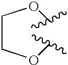 |

### Example 45

### Preparation of N-{[4′-methoxy-(1,1′-biphenyl)-4-yl]-sulfonyl}-N-methylamino-(7-methyl-1,5-dioxa-spiro[5.4]dec-7-yl)-acetic Acid

a. Methyl (1-methyl-3-oxycyclopent-1-yl)-glycinate benzophenone: Imine 26a is coupled to 3-methyl-cyclopent-2-en-1-one as described for compound 26b.

b. Ketone 45a is carried forward to the title acid as described for compound 27a-b.

### Example 46

### Preparation of N-{[4′-methoxy-(1,1′-biphenyl)-4-yl]-sulfonyl}-N-methylamino-(7-methyl-1,4-dioxa-spiro[4.4]non-7-yl)-acetic Acid

a. Methyl N-{[4′-methoxy-(1,1′-biphenyl)-4-yl]-sulfonyl}-N-methylamino-(7-methyl-1,4-dioxa-spiro[4.4]non-7-yl)-acetic acid: Ketone 45a is coupled to 1,2-ethane diol as described for compound 27a to give a white solid.

b. The ester 46a is hydrolyzed to the title acid as described for compound 1d.

### VIII. EXAMPLES—COMPOSITIONS AND METHODS OF USE

The compounds of the invention are useful to prepare compositions for the treatment of ailments associated with unwanted MP activity. The following composition and method examples do not limit the invention, but provide guidance to the skilled artisan to prepare and use the compounds, compositions and methods of the invention. In each case other compounds within the invention may be substituted for the example compound shown below with similar results. The skilled practitioner will appreciate that the examples provide guidance and may be varied based on the condition being treated and the patient.

The following abbreviations are used in this section:

EDTA: ethylenediaminetetracetic acid

SDA: synthetically denatured alcohol

USP: United States Pharmacopoeia

### Example A

A tablet composition for oral administration, according to the present invention, is made comprising:

| Component | Amount |
|---|---|
| The compound of Example 1 | 15 mg |
| Lactose | 120 mg |
| Maize Starch | 70 mg |
| Talc | 4 mg |
| Magnesium Stuart | 1 mg |

A human female subject weighing 60 kg (132 lbs), suffering from rheumatoid arthritis, is treated by a method of this invention. Specifically, for 2 years, a regimen of three tablets per day is administered orally to said subject.

At the end of the treatment period, the patient is examined and is found to have reduced inflammation, and improved mobility without concomitant pain.

### Example B

A capsule for oral administration, according to the present invention, is made comprising:

| Component | Amount (% w/w) |
|---|---|
| The compound of Example 5 | 15% |
| Polyethylene glycol | 85% |

A human male subject weighing 90 kg (198 lbs.), suffering from osteoarthritis, is treated by a method of this invention. Specifically, for 5 years, a capsule containing 70 mg of the compound of Example 3 is administered daily to said subject.

At the end of the treatment period, the patient is examined via x-ray, arthroscopy and/or MRI, and found to have no further advancement of erosion/fibrillation of the articular cartilage.

### Example C

A saline-based composition for local administration, according to the present invention, is made comprising:

| Component | Amount (% w/w) |
|---|---|
| The compound of Example 10 | 5% |
| Polyvinyl alcohol | 15% |
| Saline | 80% |

A patient having deep corneal abrasion applies the drop to each eye twice a day. Healing is speeded, with no visual sequelae.

### Example D

A topical composition for local administration, according to the present invention, is made comprising:

| Component | Composition (% w/v) |
|---|---|
| The compound of Example 21 | 0.20 |
| Benzalkonium chloride | 0.02 |
| Thimerosal | 0.002 |
| d-Sorbitol | 5.00 |
| Glycine | 0.35 |
| Aromatics | 0.075 |
| Purified water | q.s. |
| Total | 100.00 |

A patient suffering from chemical burns applies the composition at each dressing change (b.i.d.). Scarring is substantially diminished.

### Example E

An inhalation aerosol composition, according to the present invention, is made comprising:

| Component | Composition (% w/v) |
|---|---|
| Compound of Example 19 | 5.0 |
| Alcohol | 33.0 |
| Ascorbic acid | 0.1 |
| Menthol | 0.1 |
| Sodium Saccharin | 0.2 |
| Propellant (F12, F114) | q.s. |
| Total = | 100.0 |

An asthma sufferer sprays 0.01 mL via a pump actuator into the mouth while inhaling. Asthma symptoms are diminished.

### Example F

A topical opthalmic composition, according to the present invention, is made comprising:

| Component | Composition (% w/v) |
|---|---|
| Compound of Example 34 | 0.10 |
| Benzalkonium chloride | 0.01 |
| EDTA | 0.05 |
| Hydroxyethylcellulose | 0.50 |
| (NATROSOL M) | |
| Sodium metabisulfite | 0.10 |
| Sodium chloride (0.9%) | q.s. |
| Total = | 100.0 |

A human male subject weighing 90 kg (198 lbs), suffering from corneal ulcerations, is treated by a method of this invention. Specifically, for 2 months, a saline solution containing 10 mg of the compound of Example 16 is administered to said subject's affected eye twice-daily.

### Example G

A composition for parenteral administration is made comprising:

| Component | Amount |
|---|---|
| The compound of Example 2 | 100 mg/mL carrier |
| Carrier: | |
| Sodium citrate buffer with (percent | |
| by weight of carrier): | |
| lecithin | 0.48% |
| carboxymethylcellulose | 0.53 |
| povidone | 0.50 |
| methyl paraben | 0.11 |
| propyl paraben | 0.011 |

The above ingredients are mixed, forming a suspension. Approximately 2.0 mL of the suspension is administered, via injection, to a human subject with a premetastatic tumor. The injection site juxtaposes the tumor. This dosage is repeated twice daily, for approximately 30 days. After 30 days, symptoms of the disease subside, and dosage is gradually decreased to maintain the patient.

### Example H

A mouthwash composition is prepared:

| Component | % w/v |
|---|---|
| The compound of Example 41 | 3.00 |
| SDA 40 Alcohol | 8.00 |
| Flavor | 0.08 |
| Emulsifier | 0.08 |
| Sodium Fluoride | 0.05 |
| Glycerin | 10.00 |
| Sweetener | 0.02 |
| Benzoic acid | 0.05 |
| Sodium hydroxide | 0.20 |
| Dye | 0.04 |
| Water | balance |
| | to 100% |

A patient with gum disease uses 1 mL of the mouthwash thrice daily to prevent further oral degeneration.

### Example I

A lozenge composition is prepared:

| Component | % w/v |
|---|---|
| The compound of Example 37 | 0.01 |
| Sorbitol | 17.50 |
| Mannitol | 17.50 |
| Starch | 13.60 |
| Sweetener | 1.20 |
| Flavor | 11.70 |
| Color | 0.10 |
| Corn Syrup | balance |
| | to 100% |

A patient uses the lozenge to prevent loosening of an implant in the maxilla.

### Example J

### Chewing Gum Composition

| Component | w/v % |
|---|---|
| The compound of Example 6 | 0.03 |
| Sorbitol crystals | 38.44 |
| Paloja-T gum base | 20.00 |
| Sorbitol (70% aqueous solution) | 22.00 |
| Mannitol | 10.00 |
| Glycerine | 7.56 |
| Flavor | 1.00 |

A patient chews the gum to prevent loosening of dentures.

### Example K

| Components | w/v % |
|---|---|
| Compound of Example 27 | 4.0 |
| USP Water | 50.656 |
| Methylparaben | 0.05 |
| Propylparaben | 0.01 |
| Xanthan Gum | 0.12 |
| Guar Gum | 0.09 |
| Calcium carbonate | 12.38 |
| Antifoam | 1.27 |
| Sucrose | 15.0 |
| Sorbitol | 11.0 |
| Glycerin | 5.0 |
| Benzyl Alcohol | 0.2 |
| Citric Acid | 0.15 |
| Coolant | 0.00888 |
| Flavor | 0.0645 |
| Colorant | 0.0014 |

The composition is prepared by first mixing 80 kg of glycerin and all of the benzyl alcohol and heating to 65° C., then slowly adding and mixing together methylparaben, propylparaben, water, xanthan gum, and guar gum. Mix these ingredients for about 12 minutes with a Silverson in-line mixer. Then slowly add in the following ingredients in the following order: remaining glycerin, sorbitol, antifoam C, calcium carbonate, citric acid, and sucrose. Separately combine flavors and coolants and then slowly add to the other ingredients. Mix for about 40 minutes. The patient takes the formulation to prevent flare up of colitis.

### Example L

An obese human female subject, who is determined to be prone to osteoarthritis, is administered the capsule described in Example B to prevent the symptoms of osteoarthritis. Specifically, a capsule is administered daily to the subject.

The patient is examined via x-ray, arthroscopy and/or MRI, and found to have no significant advancement of erosion/fibrillation of the articular cartilage.

### Example M

A human male subject weighing 90 kg (198 lbs.), who suffers a sports injury, is administered the capsule described in Example B to prevent the symptoms of osteoarthritis. Specifically, a capsule is administered daily to the subject.

The patient is examined via x-ray, arthroscopy and/or MRI, and found to have no significant advancement of erosion/fibrillation of the articular cartilage.

All references described herein are hereby incorporated by reference.

While particular embodiments of the subject invention have been described, it will be obvious to those skilled in the art that various changes and modifications of the subject invention can be made without departing from the spirit and scope of the invention. It is intended to cover, in the appended claims, all such modifications that are within the scope of this invention.

What is claimed is:

1. A compound having a structure according to the following Formula (I): 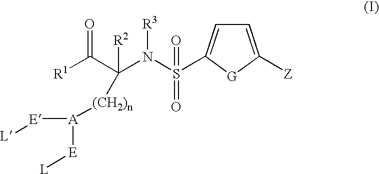
wherein:
(A) R¹ is selected from —OH and —NHOH;
(B) R² is selected from hydrogen, alkyl, alkenyl, alkynyl, heteroalkyl, haloalkyl, cycloalkyl, heterocycloalkyl, aryl, arylalkyl, heteroaryl and heteroarylalkyl; or R² and A form a ring as described in (D);
(C) R³ is selected from alkyl, alkenyl, alkynyl, heteroalkyl, haloalkyl, cycloalkyl, heterocycloalkyl, hydroxyl, alkoxy, heteroalkoxy, aryloxy, heteroaryloxy, aryl, arylalkyl, heteroaryl and heteroarylalkyl;
(D) A is a substituted or unsubstituted, monocyclic cycloalkyl having from 3 to 8 ring atoms; or A is bonded to R² where, together, they form a substituted or unsubstituted, monocyclic cycloalkyl having from 3 to 8 ring atoms;
(E) E and E′ are bonded to the same or different ring carbon atoms of A and are independently selected from a covalent bond, C₁-C₄ alkyl, aryl, heteroaryl, heteroalkyl, —O—, —S—, —N(R⁴)—, ═N, C═O, —C(═O)O—, —C(═O)N(R⁴)—, —SO₂— and —C(═S)N(R⁴)—, where R⁴ is selected from hydrogen, alkyl, alkenyl, alkynyl, heteroalkyl, haloalkyl, cycloalkyl, heterocycloalkyl, aryl, arylalkyl, heteroaryl and heteroarylalkyl; or R⁴ and L join to form a ring as described in (F)(2);
(F) (1) L and L′ are independently selected from hydrogen, alkyl, alkenyl, alkynyl, heteroalkyl, haloalkyl, aryl, arylalkyl, heteroaryl, heteroarylalkyl, cycloalkyl, heterocycloalkyl, —C(═O)R⁵, —C(═O)OR⁵, —C(═O)NR⁵R^{5′} and —SO₂R⁵, where R⁵ and R^{5′} each is independently selected from hydrogen, alkyl, alkenyl, alkynyl, heteroalkyl, haloalkyl, cycloalkyl, heterocycloalkyl, aryl, arylalkyl, heteroaryl and heteroarylalkyl; or
(2) L and R⁴ join to form an optionally substituted heterocyclic ring containing from 3 to 8 ring atoms of which from 1 to 3 are heteroatoms; or
(3) L and L′ join to form an optionally substituted cycloalkyl containing from 3 to 8 ring atoms or an optionally substituted hetercycloalkyl containing from 3 to 8 ring atoms of which from 1 to 3 are heteroatoms;
(G) G is selected from —S—, —O—, —N(R⁶)—, —C(R⁶)═C(R^{6′})—, —N═C(R⁶)— and —N═N—, where R⁶ and R^{6′} each is independently selected from hydrogen, alkyl, alkenyl, alkynyl, heteroalkyl, aryl, heteroaryl, cycloalkyl and heterocycloalkyl; and
(H) Z is selected from:
(1) cycloalkyl and heterocycloalkyl;
(2) -J-(CR⁷R^{7′})ₐR⁸ where:
(a) a is from 0 to about 4;
(b) J is selected from —C≡C—, —CH═CH—, —N═N—, —O—, —S— and —SO₂—;
(c) each R⁷ and R^{7′} is independently selected from hydrogen, alkyl, alkenyl, alkynyl, aryl, heteroalkyl, heteroaryl, cycloalkyl, heterocycloalkyl, halogen, haloalkyl, hydroxy and alkoxy; and
(d) R⁸ is selected from hydrogen, aryl, heteroaryl, alkyl, alkenyl, alkynyl, heteroalkyl, haloalkyl, heterocycloalkyl and cycloalkyl; and, if J is —C≡C— or —CH═CH—, then R⁸ may also be selected from —CONR⁹R^{9′} where (i) R⁹ and R^{9′} are independently selected from hydrogen, alkyl, alkenyl, alkynyl, haloalkyl, heteroalkyl, aryl, heteroaryl, cycloalkyl and heterocycloalkyl, or (ii) R⁹ and R^{9′}, together with the nitrogen atom to which they are bonded, join to form an optionally substituted heterocyclic ring containing from 5 to 8 ring atoms of which from 1 to 3 are heteroatoms;
(3) —NR¹⁰R^{10′} where:
(a) R¹⁰ and R^{10′} each is independently selected from hydrogen, alkyl, alkenyl, alkynyl, heteroalkyl, haloalkyl, aryl, heteroaryl, cycloalkyl, heteroalkyl and —C(═O)-Q-(CR¹¹R^{11′})_{b}R¹² where:
(i) b is from 0 to about 4;
(ii) Q is selected from a covalent bond and —N(R¹³)—; and
(iii) each R¹¹ and R^{11′} is independently selected from hydrogen, alkyl, alkenyl, alkynyl, aryl, heteroalkyl, heteroaryl, cycloalkyl, heterocycloalkyl, halogen, haloalkyl, hydroxy and alkoxy; either (A) R¹² and R¹³ each is independently selected from hydrogen, alkyl, alkenyl, alkynyl, heteroalkyl, haloalkyl, aryl, heteroaryl, cycloalkyl and heterocycloalkyl, or (B) R¹² and R¹³, together with the atoms to which they are bonded, join to form an optionally substituted heterocyclic ring containing from 5 to 8 ring atoms of which from 1 to 3 are heteroatoms; or R¹⁰ and R¹³, together with the nitrogen atoms to which they are bonded, join to form an optionally substituted heterocyclic ring containing from 5 to 8 ring atoms of which from 2 to 3 are heteroatoms; or
(b) R¹⁰ and R^{10′}, together with the nitrogen atom to which they are bonded, join to form an optionally substituted heterocyclic ring containing from 5 to 8 ring atoms of which from 1 to 3 are heteroatoms; and
(4) 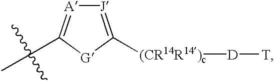
where:
(a) A′ and J′ are independently selected from —CH— and —N—;
(b) G′ is selected from —S—, —O—, —N(R¹⁵)—, —C(R¹⁵)═C(R^{15′})—, —N═C(R¹⁵)— and —N═N—, where R¹⁵ and R^{15′} each is independently selected from hydrogen, alkyl, alkenyl, alkynyl, heteroalkyl, aryl, heteroaryl, cycloalkyl and heterocycloalkyl;
(c) c is from 0 to about 4;
(d) each R¹⁴ and R^{14′} is independently selected from hydrogen, alkyl, alkenyl, alkynyl, aryl, heteroalkyl, heteroaryl, cycloalkyl, heterocycloalkyl, halogen, haloalkyl, hydroxy and alkoxy;
(e) D is selected from a covalent bond, —O—, —SO_{d}—, —C(═O)—, —C(═O)N(R¹⁶)—, —N(R¹⁶)—, and —N(R¹⁶)C(═O)—; where d is from 0 to 2 and R¹⁶ is selected from hydrogen, alkyl, alkenyl, alkynyl, aryl, heteroaryl, heteroalkyl, heteroaryl, cycloalkyl, heterocycloalkyl and haloalkyl; and
(f) T is —(CR¹⁷R^{17′})ₑ—R¹⁸ where e is from 0 to about 4; each R¹⁷ and R^{17′} is independently selected from hydrogen, alkyl, alkenyl, alkynyl, aryl, heteroalkyl, heteroaryl, cycloalkyl, heterocycloalkyl, halogen, haloalkyl, hydroxy, alkoxy and aryloxy; and R¹⁸ is selected from hydrogen, alkyl, alkenyl, alkynyl, halogen, heteroalkyl, haloalkyl, aryl, heteroaryl, cycloalkyl and heterocycloalkyl; or R¹⁷ and R¹⁸, together with the atoms to which they are bonded, join to form an optionally substituted heterocyclic ring containing from 5 to 8 atoms of which 1 to 3 are heteroatoms; or R¹⁶ and R¹⁸, together with the atoms to which they are bonded, join to form an optionally substituted heterocyclic ring containing from 5 to 8 atoms of which 1 to 3 are heteroatoms;
or an optical isomer, diastereomer or enantiomer for Formula (I), or a pharmaceutically-acceptable salt, or biohydrolyzable amide, ester, or imide thereof.

2. The compound of claim 1 wherein R¹ is —OH.

3. The compound of claim 1 wherein R¹ is —NHOH.

4. The compound of claim 1 wherein A is substituted or unsubstituted cyclopentane or cyclohexane.

5. The compound of claim 4 wherein A is substituted or unsubstituted cyclohexane.

6. The compound of claim 1 wherein E and E′ are bonded to the same ring carbon atom of A and are independently selected from —O— and —S—, and wherein L and L′ join to form an optionally substituted hetercycloalkyl containing from 3 to 8 ring atoms of which 2 are heteroatoms.

7. The compound of claim 1 wherein E′ is a covalent bond, L′ is hydrogen, and E is selected from —O—, —S—, NR⁴ and —SO₂—.

8. The compound of claim 7 wherein (i) L is selected from hydrogen, alkyl, heteroalkyl, aryl, arylalkyl, heteroaryl, heteroarylalkyl, heterocycloalkyl, —C(═O)R⁵, —C(═O)OR⁵, —C(═O)NR⁵R^{5′} and —SO₂R⁵ or (ii) L and R⁴ join to form an optionally substituted heterocyclic ring containing from 3 to 8 ring atoms of which from 1 to 3 are heteroatoms.

9. The compound of claim 1 wherein n=0.

10. The compound of claim 1 wherein G is selected from —S— and —C(R⁶)═C(R^{6′})—.

11. The compound of claim 1 wherein Z is —NR¹⁰R^{10′} where R¹⁰ is hydrogen and R^{10′} is —C(O)-Q-(CR¹¹R^{11′})_{b}R¹² where b is 0, Q is selected from a covalent bond and —N(R¹³)—, and R¹² is selected from aryl, heteroaryl, cycloalkyl, and heterocycloalkyl, or R¹² and R¹³, together with the nitrogen atom to which they are bonded, join to form an optionally substituted heterocyclic ring containing 5 or 6 ring atoms of which from 1 or 2 are heteroatoms.

12. The compound of claim 1 wherein Z is 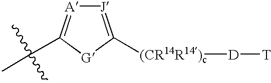
where A′ and J′ are CH—; G′ is —N═C(R¹⁵)— or —C(R¹⁵)═C(R^{15′})—, where R¹⁵ and R^{15′} each is independently selected from hydrogen and lower alkyl; c is 0; D is a covalent bond or —O—; and T is —(CR¹⁷R^{17′})ₑ—R¹⁸ where e is 0 and R¹⁸ is selected from lower alkyl, lower heteroalkyl, halogen and aryl.

13. The compound of claim 1 wherein R³ is alkyl, heteroalkyl, cycloalkyl, heterocycloalkyl, aryl, arylalkyl, heteroaryl and heteroarylalkyl.

14. A compound having a structure according to the following Formula (I): 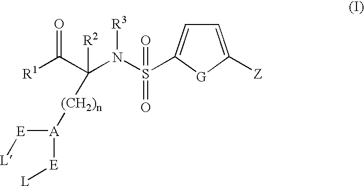
wherein:
(A) R¹ is selected from —OH and —NHOH;
(B) R² is selected from hydrogen and alkyl; or R² and A form a ring as described in (D);
(C) R³ is alkyl, heteroalkyl, cycloalkyl, heterocycloalkyl, aryl, arylalkyl, heteroaryl and heteroarylalkyl;
(D) A is a substituted or unsubstituted, monocyclic cycloalkyl having 5 or 6 ring atoms; or A is bonded to R² where, together, they form a substituted or unsubstituted, monocyclic cycloalkyl having 5 or 6 ring atoms;
(E) E and E′ are bonded to the same or different ring carbon atoms of A; E is selected from —O—, —S—, NR⁴ and —SO₂—, where R⁴ is selected from hydrogen, alkyl, heteroalkyl, haloalkyl, cycloalkyl, heterocycloalkyl, aryl, arylalkyl, heteroaryl and heteroarylalkyl; and E′ is a covalent bond;
(F) (1) L is selected from hydrogen, alkyl, heteroalkyl, aryl, arylalkyl, heteroaryl, heteroarylalkyl, heterocycloalkyl, —C(═O)R⁵, —C(═O)OR⁵, —C(═O)NR⁵R^{5′} and —SO₂R⁵, where R⁵ is selected from hydrogen, alkyl, heteroalkyl, cycloalkyl, heterocycloalkyl, aryl, arylalkyl, heteroaryl and heteroarylalkyl; and L′ is hydrogen; or
(2) L and R⁴ join to form an optionally substituted heterocyclic ring containing from 3 to 8 ring atoms of which from 1 to 3 are heteroatoms; or
(3) L and L′ join to form an optionally substituted cycloalkyl containing from 3 to 8 ring atoms or an optionally substituted hetercycloalkyl containing from 3 to 8 ring atoms of which from 1 to 3 are heteroatoms;
(F) G is selected from —S— and —C(R⁶)═C(R^{6′})—, where R⁶ and R^{6′} each is independently selected from hydrogen and alkyl;
(H) Z is selected from:
(1) —NR¹⁰R^{10′} where:
(a) R¹⁰ is hydrogen and R^{10′} is —C(═O)-Q-(CR¹¹R^{11′})_{b}R¹² where:
(i) b is 0;
(ii) Q is selected from a covalent bond and —N(R¹³)—; and
(iii) (A) R¹² and R¹³ each is independently selected from hydrogen, alkyl, alkenyl, alkynyl, heteroalkyl, haloalkyl, aryl, heteroaryl, cycloalkyl, and heterocycloalkyl, or (B) R¹² and R¹³, together with the atoms to which they are bonded, join to form an optionally substituted heterocyclic ring containing from 5 to 8 ring atoms of which from 1 to 3 are heteroatoms; or
(b) R¹⁰ and R^{10′}, together with the nitrogen atom to which they are bonded, join to form an optionally substituted heterocyclic ring containing 5 or 6 ring atoms of which from 1 or 2 are heteroatoms; and
(2) 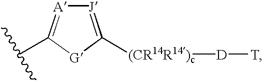
where:
(a) A′ and J′ both are —CH—;
(b) G′ is selected from —C(R¹⁵)═C(R^{15′})— and —N═C(R¹⁵)—, where R¹⁵ and R^{15′} each is independently selected from hydrogen and lower alkyl;
(c) c is from 0;
(d) D is selected from is a covalent bond or —O—; and
(e) T is —(CR¹⁷R^{17′})ₑ—R¹⁸ where e is 0; and R¹⁸ is selected from lower alkyl, lower heteroalkyl, halogen and aryl;
or an optical isomer, diastereomer or enantiomer for Formula (I), or a pharmaceutically-acceptable salt, or biohydrolyzable amide, ester, or imide thereof.

15 The compound of claim 1, selected from the group consisting of:
N-{[4′-Methoxy-(1,1′-biphenyl)-4-yl]-sulfonyl-N-methyl-amino}-(4-hydroxycyclohexan-1-yl)-acetic acid;
(R)-N-{[4′-methoxy-(1,1′-biphenyl)-4-yl]-sulfonyl}-N-methylamino-(1,5-dioxa-spiro[5.5]undec-9-yl)-acetic acid;
(R)—N-{[4′-bromo-(1,1′-biphenyl)-4-yl]-sulfonyl}-N-methylamino-(1,5-dioxa-spiro[5.5]undec-9-yl)-acetic acid;
(1,4-Dioxa-spiro[4.5] dec-8-yl)-N-{[4′-Methoxy-(1,1′-biphenyl)-4-yl]-sulfonyl}-N-methylamino-acetic acid;
(1,4-Dioxa-spiro[4.5]dec-8-yl)-N-{[4′-Methoxy-(1,1′-biphenyl)-4-yl]-sulfonyl}-N-(pyridin-4-yl-methyl)-amino-acetic acid;
(1,4-Dioxa-spiro[4.5]dec-8-yl)-N-{[4′-Methoxy-(1,1′-biphenyl)-4-yl]-sulfonyl}-N-(2-methoxyethyl)-amino-acetic acid;
(1,4-Dioxa-spiro[4.5]dec-8-yl)-N-{[4′-Methoxy-(1,1′-biphenyl)-4-yl]-sulfonyl}-N-(N-morpholinoethyl)-amino-acetic acid;
2-(1,4-Dioxa-spiro[4.5]dec-8-yl)-2N-{[4′-Methoxy-(1,1′-biphenyl)-4-yl]-sulfonyl}-methylamino-propionic acid;
[Spiro-(1,3-benzodioxole-2,1′-cyclohex-4′-yl]-N-{[4′-Methoxy-(1,1′-biphenyl)-4-yl]-sulfonyl}-N-methylamino-acetic acid;
(7-methylthio-1,4-Dioxaspiro[4.5]dec-7-yl)-N-{[4′-Methoxy-(1,1′-biphenyl)-4-yl]-sulfonyl}-N-methylamino-acetic acid;
N-{[4′-Methoxy-(1,1′-biphenyl)-4-yl]-sulfonyl-N-methylamino}-[4-(N-benzylamino)-cyclohexan-1-yl]-acetic acid;
N-{[4′-Methoxy-(1,1′-biphenyl)-4-yl]-sulfonyl-N-methylamino}-[4-(N-benzyl-N-acetyamino)-cyclohexan-1-yl]-acetic acid;
N-{[4′-Methoxy-(1,1′-biphenyl)-4-yl]-sulfonyl-N-methylamino}-[4-(N-benzyl-N-methanesulfonylamino)-cyclohex-1-yl]-acetic acid;
N-{[4′-Methoxy-(1,1′-biphenyl)-4-yl]-sulfonyl-N-methylamino}-(4-N-methoxymethylacetylamino-cyclohexan-1-yl)-acetic acid;
N-{[4′-Methoxy-(1,1′-biphenyl)-4-yl]-sulfonyl-N-methylamino}-(4-N-methoxymethylacetyl-N-methylamino-cyclohexan-1-yl)-acetic acid;
N-{[4′-Methoxy-(1,1′-biphenyl)-4-yl]-sulfony-N-methyl-lamino}-(4-N-acetyl-N-methylamino-cyclohexan-1-yl)-acetic acid;
N-{[4′-Methoxy-(1,1′-biphenyl)-4-yl]-sulfonyl-N-methyl-amino}-(4-N-dimethylacetyl-N-methyl-aminocyclohexan-1-yl)-acetic acid;
N-{[4′-Methoxy-(1,1′-biphenyl)-4-yl]-sulfonyl-N-methylamino}-[4-(morpholin-4N-yl)-cyclohexan-1-yl]-acetic acid;
N-{[4′-Methoxy-(1,1′-biphenyl)-4-yl]-sulfonyl-N-methylamino}-[4-(2-oxopyrrolidin-1N-yl)-cyclohexan-1-yl]-acetic acid;
N-{[4′-Methoxy-(1,1′-biphenyl)-4-yl]-sulfonyl-N-(2-methoxyethyl)-amino}-[4-(2-oxopyrrolidin-1N-yl)-cyclohexan-1-yl]-acetic acid;
N-{[4′-Methoxy-(1,1′-biphenyl)-4-yl]-sulfonyl-N-methylamino}-[4-(oxazolidin-2-one-3N-yl)-cyclohexan-1-yl]-acetic acid;
N-{[4′-Methoxy-(1,1′-biphenyl)-4-yl]-sulfonyl-N-methylamino}-[4-([1,3]-oxazinan-2-one-3N-yl)-cyclohexan-1-yl]-acetic acid;
N-{[4′-Methoxy-(1,1′-biphenyl)-4-yl]-sulfonyl-N-methylamino}-[4-(2-oxomorpholin-1N-yl)-cyclohexan-1-yl]-acetic acid;
N-{[4′-Methoxy-(1,1′-biphenyl)-4-yl]-sulfonyl-N-methylamino}-[4-(3N-methyl-hydantoin-1N-yl)-cyclohexan-1-yl]-acetic acid;
N-{[4′-Methoxy-(1,1′-biphenyl)-4-yl]-sulfonyl-N-methylamino}-[4-(□-sultam-1N-yl)-cyclohexan-1-yl]-acetic acid;
N-{[4′-methoxy-(1,1′-biphenyl)-4-yl]-sulfonyl}-N-methyl-amino-(3-hydroxycyclohexan-1-yl)-acetic acid;
N-{[4′-methoxy-(1,1′-biphenyl)-4-yl]-sulfonyl}-N-methylamino-(1,5-dioxa-spiro[5.5]undec-7-yl)-acetic acid;
N-{[4′-bromo-(1,1′-biphenyl)-4-yl]-sulfonyl}-N-methyl-amino-(1,5-dioxa-spiro[5.5]undec-7-yl)-acetic acid;
N-{[4′-methoxy-(1,1′-biphenyl)-4-yl]-sulfonyl}-N-benzylamino-(1,5-dioxa-spiro[5.5]undec-7-yl)-acetic acid;
N-{[4′-methoxy-(1,1′-biphenyl)-4-yl]-sulfonyl}-N-methylamino-(7-methyl-1,5-dioxa-spiro[5.5]undec-7-yl)-acetic acid;
N-{[4′-methoxy-(1,1′-biphenyl)-4-yl]-sulfonyl}-N-benzylamino-(7-methyl-1,5-dioxa-spiro[5.5]undec-7-yl)-acetic acid;
N-{[4′-methoxy-(1,1′-biphenyl)-4-yl]-sulfonyl}-N-methyl-amino-(3-benzyloxycyclohexan-1-yl)-acetic acid;
N-{[4′-Methoxy-(1,1′-biphenyl)-4-yl]-sulfonyl-N-methylamino}-[3-(N-benzylamino)-cyclohexan-1-yl]-acetic acid;
N-{[4′-Methoxy-(1,1′-biphenyl)-4-yl]-sulfonyl-N-methylamino}-[3-(N-benzyl-N-acetyamino)-cyclohexan-1-yl]-acetic acid;
N-{[4′-Methoxy-(1,1′-biphenyl)-4-yl]-sulfonyl-N-methylamino}-(3-N-(2-methoxyethoxycarbonyl)-amino-cyclohexan-1-yl)-acetic acid;
N-{[4′-Methoxy-(1,1′-biphenyl)-4-yl]-sulfonyl-N-methylamino}-(3-N-methyl-N-(2-methoxyethoxycarbonyl)-amino-cyclohexan-1-yl)-acetic acid;
N-{[4′-Methoxy-(1,1′-biphenyl)-4-yl]-sulfonyl-N-methylamino}-[3-(morpholin-4N-yl)-cyclohexan-1-yl]-acetic acid;
N-{[4′-Methoxy-(1,1′-biphenyl)-4-yl]-sulfonyl-N-methylamino}-[3-(2-oxopyrrolidin-1N-yl)-cyclohexan-1-yl]-acetic acid;
N-{[4′-Methoxy-(1,1′-biphenyl)-4-yl]-sulfonyl-N-methylamino}-[3-(2-oxomorpholin-1N-yl)-cyclohexan-1-yl]-acetic acid;
N-{[4′-Methoxy-(1,1′-biphenyl)-4-yl]-sulfonyl-N-methylamino}-[3-(3N-methyl-hydantoin-1N-yl)-cyclohexan-1-yl]-acetic acid;
N-{[4′-Methoxy-(1,1′-biphenyl)-4-yl]-sulfonyl-N-methylamino}-[3-(□-sultam-1N-yl)-cyclohexan-1-yl]-acetic acid;
N-{[4′-Methoxy-(1,1′-biphenyl)-4-yl]-sulfonyl-N-methylamino}-[3-(□-sultam-1N-yl)-cyclohexan-1-yl]-acetic acid;
N-{[4′-Methoxy-(1,1′-biphenyl)-4-yl]-sulfonyl-N-methylamino}-[3-(oxazolidin-2-one-3N-yl)-cyclohexan-1-yl]-acetic acid;
N-{[4′-Methoxy-(1,1′-biphenyl)-4-yl]-sulfonyl-N-methylamino}-[1-methyl 3-(oxazolidin-2-one-3N-yl)-cyclohexan-1-yl]-acetic acid;
N-{[4′-methoxy-(1,1′-biphenyl)-4-yl]-sulfonyl}-N-methylamino-(7-methyl-1,5-dioxa-spiro[5.4]dec-7-yl)-acetic acid; and
N-{[4′-methoxy-(1,1′-biphenyl)-4-yl]-sulfonyl}-N-methylamino-(7-methyl-1,4-dioxa-spiro[4.4]non-7-yl)-acetic acid.

16. A pharmaceutical composition comprising:
(a) a safe and effective amount of a compound of claim 1; and
(b) a pharmaceutically-acceptable carrier.

17. A pharmaceutical composition comprising:
(a) a safe and effective amount of a compound of claim 14; and
(b) a pharmaceutically-acceptable carrier.

18. A method for treating a metalloprotease related disorder in a mammalian subject, the method comprising administering to said subject a safe and effective amount of a compound of claim 1.

19. The method of claim 18, wherein the disorder is chosen from the group consisting of arthritis, cancer, cardiovascular disorders, skin disorders, ocular disorders, inflammation and gum disease.

20. The method of claim 18, wherein the disorder is arthritis, and is chosen from the group consisting of osteoarthritis and rheumatoid arthritis.

21. The method of claim 18, wherein the disorder is cancer, and the treatment prevents or arrests tumor growth and metastasis.

22. The method of claim 18, wherein the disorder is a cardiovascular disorder selected from the group consisting of dilated cardiomyopathy, congestive heart failure, atherosclerosis, plaque rupture, reperfusion injury, ischemia, chronic obstructive pulmonary disease, angioplasty restenosis and aortic aneurysm.

23. The method of claim 18, wherein the disorder is an ocular disorder, and is chosen from the group consisting of corneal ulceration, lack of corneal healing, macular degeneration, retinopathy and pterygium.

24. The method of claim 18, wherein the disorder is gum disease, and is chosen from the group consisting of periodontal disease and gingivitis.

25. The method of claim 18, wherein the disorder is a skin a disorder chosen from the group consisting of wrinkle repair and prevention, U.V. skin damage, epidermolysis bullosa, psoriasis, sclerodema, atopic dermatitis and scarring.

26. A method of claim 18, wherein said inflammatory condition is chosen from the group consisting of inflammatory bowel disease, Crohn's Disease, ulcerative colitis, pancreatitis, diverticulitis, acne inflammation, bronchitis, arthritis and asthma.

27. The method of claim 18, wherein the disorder is multiple sclerosis.

28. The method of claim 18, wherein the disorder is the loosening of prosthetic devices.

29. The method of claim 28, wherein the loosening of prosthetic devices is selected from joint replacements and dental prosthesis.

30. The method of claim 18, wherein the disorder is selected from chronic heart failure, myocardial infarction and progressive ventricular dilation.
